(19) United States
(12) Patent Application Publication
(10) Pub. No.: US 2023/0151407 A1
(43) Pub. Date: May 18, 2023

(54) A METHOD FOR MULTIPLEXED DETECTION OF A PLURALITY OF TARGET BIOMOLECULES

(71) Applicant: APLEX BIO AB, Solna (SE)

(72) Inventors: Umear Naseem, Solna (SE); Ruben Rafael Gonçalves Soares, Solna (SE)

(21) Appl. No.: 17/917,030

(22) PCT Filed: Apr. 6, 2021
(86) PCT No.: PCT/SE2021/050306

§ 371 Date: Oct. 5, 2022

(30) Foreign Application Priority Data
Apr. 6, 2020 (SE) 2050384-3

(51) Int. Cl. C12Q 1/6816; C09K 11/02; C09K 11/59; G01N 21/64

(52) U.S. Cl. CPC C12Q 1/6816; C09K 11/025; C09K 11/59; G01N 21/6428; G01N 21/6456; C12Q 2600/16; C12Q 1/70

(57) ABSTRACT

Described is a method for multiplexed detection of a plurality of target biomolecules having at least one detection target using optical encoding. The method includes steps:
a. providing one or more nanoparticle types having a plurality of nanoparticles, each nanoparticle having a coating that provides binding affinity of the nanoparticle to a type-specific detection target, wherein each nanoparticle has a plurality of fluorophores that generates a signal which is unique for each nanoparticle type;
b. providing a sample having a plurality of target biomolecules;
c. contacting the sample with the plurality of nanoparticle types, thereby allowing the nanoparticles to bind with the detection targets of the target biomolecules;
d. optically decoding the fluorophore signals emitted by the nanoparticle of the nanoparticle type bound to the detection target of the target biomolecules by measuring wavelength and intensity of the emitted signals, thereby detecting the presence and identity of the target biomolecules.

### TECHNICAL FIELD

The present disclosure relates to a method for multiplexed detection of a plurality of target biomolecules. More specifically, the disclosure relates to a method for multiplexed detection of a plurality of target biomolecules as defined in the introductory parts of claim 1, and a kit of parts as defined in claim 15.

### BACKGROUND ART

Many bioanalytical technologies typically use fluorescent methods to allow for multiplexed (more than one) detection of various targets. Several applications benefit from doing this such as histology, flow cytometry, fundamental cellular and molecular protocols, fluorescence in situ hybridization, DNA sequencing, immuno assays, binding assays etc. While it is possible to detect more than one target simultaneously, using fluorophores still severely limits the ability to multiplex. For the in situ hybridization application especially new technologies are emerging in order to increase the magnitude of multiplexing capacity focused on barcode decoding via in situ sequencing schemes.

The use of multiple fluorophores (dyes) is limited by its physical properties, where each dye has a certain spectral shape of its absorbance and emission (about 100 nm broad for organic dyes). Due to this, when multiple fluorophores are used simultaneously their emissions need to be well separated. This causes a limitation in the amount of dyes that can be used simultaneously before the emission overlaps (spectral overlap) such that it becomes difficult to distinguish one dye from another (typically less than 5-10 dyes simultaneously).

In order to overcome this, in situ sequencing technologies are emerging (Strell et al. (FEBS J. (2018), p. 14435 (Placing RNA in context and space-methods for spatially resolved transcriptomics)) and Lein et al. (Science (2017) 358, 64-69 (The promise of spatial transcriptomics for neuroscience in the era of molecular cell typing.)) are two relevant articles in this context). These make use of molecular DNA barcodes that represent each a unique target that needs to be decoded for the successful detection to be completed (targeted method), or alternatively doing the direct sequencing of the target in case of RNA/DNA (non-targeted). The process of decoding involves the use of DNA sequencing chemistry (addition of signal and removal), the iterative process of stepping one DNA base at a time and repeating this the amount of times necessary depending on the level of multiplexing desired, maintaining the integrity and location of sample during this iterative process, image acquisition between each iteration, and finally the image analysis to puzzle and align all images together to thereafter decide on the identity of each target. The whole process therefore takes much longer time than that of one-step (direct) detection methods (such as conventional fluorescent in situ hybridization), and typically requires trained personnel and/or robust hardware that integrates chemical steps with imaging steps resulting in high costs. Furthermore, the process of doing several steps iteratively also makes the sequencing approach sensitive to errors or mistakes, as it is enough for a sub-part of the process to go wrong for the whole decoding to fail altogether. The sequencing process is therefore more complicated, sensitive to errors, expensive, and time consuming in comparison to a direct approach.

There is thus a need for improved methods for multiplexed detection of a plurality of target biomolecules, taking into account the problems of the existing solutions.

### SUMMARY OF THE INVENTION

It is an object of the present disclosure to mitigate, alleviate or eliminate one or more of the above-identified deficiencies and disadvantages in the prior art and solve at least the above mentioned problem.

According to a first aspect of the invention there is provided a method for multiplexed detection of a plurality of target biomolecules using optical encoding, wherein each target biomolecule has at least one detection target, comprising the steps of:
a. providing a plurality of nanoparticle types comprising a plurality of nanoparticles, each nanoparticle having a coating that provides binding affinity of the nanoparticle to a type-specific detection target, and wherein each nanoparticle comprises a plurality of fluorophores that generates a signal which is unique for each nanoparticle type;
b. providing a sample comprising a plurality of target biomolecules;
c. contacting the sample with the plurality of nanoparticle types, thereby allowing the nanoparticles to bind with the detection targets of the target biomolecules; and
d. optically decoding the fluorophore signals emitted by the nanoparticle of the nanoparticle type bound to the detection target of the target biomolecules by measuring the wavelength and intensity of the emitted signal, thereby detecting the presence and identity of the target biomolecules.

Hereby, a method of simultaneously detecting a significant plurality (e.g. 50-100) of detection targets in situ using optically encoded nanoparticles in a one-step direct manner is provided. This can be achieved by tailoring the nanoparticles of each nanoparticle type to achieve distinct and precise incorporation of fluorophores in well-defined nanoparticles and thereby optically encoding such.

As used herein, the term “a plurality” refers to at least two.

As used herein, when referring to nanoparticles comprising a plurality of fluorophores, this means that the nanoparticles comprise at least two different fluorophores exhibiting different emission wavelengths and/or intensities. For example, the nanoparticles of each nanoparticle type comprise at least two, such as at least three, four or five different fluorophores.

The nanoparticles can in addition provide protection for the fluorophores to prevent bleaching. This is important because the number of combinations becomes limited if the intensity distributions overlap with each other.

Furthermore, it is possible to utilize semiconductor fluorophores such as quantum dots to achieve a greater range of combinations, either by incorporating multiple such or in combination with organic fluorophores.

The number of combinations becomes n^{m}−1, where n is the number of intensity levels and m the number of colors. It follows that the number of combinations scales greater with the number of colors rather than the number of intensity levels. Using semiconductor fluorophores it is possible to achieve both a narrower emission spectrum (enabling more colors in parallel before spectral overlap becomes a problem), as well as fitting in more colors in a spectrum together with organic fluorophores due to the possibility of having semiconductor fluorophores with very large stoke shifts.

By using nanoparticles it is also possible to alter the properties of individual fluorophores when incorporated in an organized matter in a particle where distance between the fluorophores can be carefully controlled, such that they can be coupled energetically and act as waveguides/antennas, such as Förster resonance energy transfer (FRET) or excitation energy transfer (EET). This allows for further flexibility in achieving a higher number of concentration levels and/or higher number of color combinations, leading ultimately to a higher number of multiplexity.

In an embodiment, the nanoparticle types are in suspension, that is to say the nanoparticles of the nanoparticle types are in suspension.

The invention allows for the process of nanoparticles with a certain unique identity binding to a detection target (DNA/RNA/protein) in situ, where the size of the nanoparticles allows for the penetration of cells and that the nanoparticles can immobilize onto the target while the excess is washed away. Preferably, the detection target is designed such that a plurality of nanoparticles can attach within at least one optically resolvable pixel forming a cluster of nanoparticles in the spot of detection target, such that the signal intensity becomes higher in this cluster compared to the signal from single nanoparticles that may bind non-specifically to the surrounding, allowing for a localized signal to be detected upon target binding, where the unique identity of the nanoparticle encodes for the target identity. Typically this may be done by performing target amplification prior to decoding, such as rolling-circle amplification (RCA) or other means of localized/clonal amplification (hairpin chain reaction etc.), or by designing probes in a manner similar to traditional fluorescent in situ hybridization (FISH) techniques such that they can form a localized cluster.

The invention also allows for the process of nanoparticles with a certain unique identity binding to a detection target or biomolecule target (DNA/RNA/protein) in situ, where the size of the nanoparticles allows for the penetration of cells and that the nanoparticles can immobilize to the biomolecule target while the excess is washed away. Alternatively, the biomolecule targets are not in cells but are instead immobilized in a 2D or 3D matrix of material, and are still considered to be in situ. For example, the target biomolecules can be immobilized onto the surface of a microscope slide, or in a 3D polymer network, or within tissue where furthermore optionally most of the tissue components except the biomolecule targets are removed. Preferably, the target biomolecule is designed such that a plurality of nanoparticles can attach within at least one optically resolvable pixel forming a cluster of nanoparticles in the spot of detection target or target biomolecule comprising detection targets, such that the signal intensity becomes higher in this cluster compared to the signal from single nanoparticles that may bind non-specifically to the surrounding, allowing for a localized signal to be detected upon target binding, and where the unique identity of the nanoparticles that encodes for the target identity can be read/decoded in an isolated manner because the plurality of nanoparticles that bind to one biomolecule target are of the same nanoparticle type. Such signal would therefore be spatially resolved, because each signal identity encoding for the target identity can be read/decoded in an isolated manner with respect to the nanoparticle type, without a significant interference of another nanoparticle type signal within this spatially resolved spot or volume, meaning that even if there may be a small interference by unspecifically bound nanoparticles from another nanoparticle type in the localized spot of signal, the signal from the specifically bound nanoparticles from the correct nanoparticle type is strong enough to decode the identity with confidence. Typically this may be done by performing target amplification prior to decoding, such as rolling-circle amplification (RCA) or other means of localized/clonal amplification (hairpin chain reaction etc.), or by designing probes in a manner similar to traditional fluorescent in situ hybridization (FISH) techniques such that they can form a localized cluster that is spatially resolved, enlarging the size of the original biomolecule target and thereby allowing multiple nanoparticles of the same nanoparticle type to bind to the biomolecule target. Such amplifications methods are sometimes also commonly referred to as clonal amplification methods.

That is to say, the invention allows for the process of nanoparticles with a certain unique identity to bind to a detection target or biomolecule target in a sample (DNA/RNA/protein), where the nanoparticles can immobilize onto the detection target allowing for excess non-bound nanoparticles to be washed away from the sample after the contacting step.

In an embodiment the method of the invention may be performed in-situ or ex-situ. The term in-situ refers to the sample contacting the nanoparticles after being immobilized on a 2D or 3D matrix of material. The term ex-situ refers to sample contacting the nanoparticles in an homogeneous solution.

As used herein, the term “2D or 3D matrix of material” refers to a solid phase made of glass, or plastics, such as polystyrene, polyethylene, polymethylmethacrylate, cyclic olefin copolymer, agarose, biodegradable polymers (such as polylactide, poly(glycolides), poly(ε-caprolactone)) or combinations thereof. The solid phase may be coated with biomolecules such as proteins and/or nucleic acids to improve the immobilization of the sample and/or passivation of the surface. Alternatively the solid phase may not be coated.

As used herein, the term “sample” refers to any sample containing a target biomolecule (e.g. a plurality of target biomolecules). For example, a sample may be a biological sample, such as a body fluid sample, a tissue sample or a single cell. The sample may further comprise a mixture of biomolecules including proteins and/or nucleic acids in an homogeneous solution or immobilised on a “2D” or “3D” matrix of material via affinity, electrostatic, covalent, or Van der Waals interactions, or combinations thereof.

As used herein, the term “detection target” refers to any target in the sample to which the nanoparticle(s) can bind.

In an embodiment the detection target is a polynucleotide sequence. By the term “polynucleotide sequence” we include any biopolymer composed of nucleotide monomers in a chain, for example DNA and/or cDNA and/or RNA and/or a protein.

Preferably, the detection target is designed such that a plurality of nanoparticles can attach within at least one optically resolvable pixel forming a cluster of nanoparticles in the spot of detection target, such that the signal intensity becomes higher in this cluster compared to the signal from single nanoparticles that may bind non-specifically to the surrounding, allowing for a localized signal to be detected upon target binding, where the unique identity of the nanoparticle encodes for the target identity. Typically this may be done by performing target amplification prior to decoding, such as rolling-circle amplification (RCA), Polymerase Chain Reaction (PCR), Reverse Transcriptase Polymerase Chain Reaction (RT-PCR), Loop mediated isothermal amplification (LAMP) or other means of localized/clonal amplification (hairpin chain reaction etc.), or by designing probes in a manner similar to traditional fluorescent in situ hybridization (FISH) techniques such that they can form a localized cluster. These methods may be based on the use of exonuclease, endonuclease, transposase or CRISPR/Cas-based enzymatic activity prior to amplification. The amplification procedure may also comprise a combination of two or more of the techniques above.

According to some embodiments, each nanoparticle type is optically encoded by (i) incorporating precisely controlled ratios of a plurality of fluorophores, thereby controlling the emission wavelength and intensity from the nanoparticle type, or (ii) altering the properties of the fluorophores affecting their emission intensities.

Put in another way, each nanoparticle type may be optically encoded by incorporating predetermined amounts of a plurality of fluorophores into and/or onto the nanoparticle, thereby arriving at a nanoparticle type that exhibits at least one, for example at least two, three four or five, specific emission wavelength(s) and intensity(ies).

By the term “incorporating” as used herein we refer to nanoparticles that are loaded with the plurality of fluorophores. That is to say, in an embodiment the nanoparticles are loaded with the fluorophores. The loading may be on the surface of the nanoparticles, or within the matrix of the nanoparticles themselves, or within pores of the nanoparticle and/or the nanoparticles may encapsulate the fluorophores.

By “loading within the matrix of the nanoparticles themselves”, we refer to incorporating the fluorophores into the nanoparticles as they are synthesized, thus leading to the fluorophores being embedded within the matrix of the nanoparticles.

By encapsulating the fluorophores in the matrix of the nanoparticles, the process of bleaching of the fluorophores can be slowed down. For example, bleaching usually is faster in the presence of oxygen, so slowing the diffusion of oxygen into the particle is one way to slow bleaching. The other aspect is that the rate of photobleaching is dependent on the environment, for example solvent conditions. This way, the “solvent conditions” inside the particle can be made different from outside the particle, for example when encapsulating the fluorophores in a non-polar matrix such as a polymer matrix.

Thus, the invention allows for the use of optically encoded nanoparticles that have been synthesized with the incorporation of precise controlled intensity levels of dyes (e.g. fluorophores) such that each the combination of levels (ratios) represents a unique identity, enabling higher number of multiple identities compared to single colors alone. The number of combinations becomes n^{m}−1, where n is the number of intensity levels and m the number of colors.

By the term “ratios” as used herein we include reference to the dyes/fluorophores being incorporated in the nanoparticles in a predetermined amount.

According to some embodiments, the binding affinity of the coating is provided by a detection probe X attached via a linker to the nanoparticle. In an embodiment the detection probe X is a nucleic acid molecule, an antigen, an antibody or combinations thereof.

In an embodiment, the linker is not present and the probe X is bound directly to the nanoparticle surface. The “detection probes” refer to molecules that bind specifically to a target molecule or a group of target molecules and include oligonucleotides with deoxyribose and/ribose bases, xeno nucleic acid, such as locked nucleic acids (LNA), a glycol nucleic acid (GNA), a threose nucleic acid (TNA), a phosphorodiamidate Morpholino oligomer (PMO), peptide nucleic acid (PNA), antibodies, antibody fragments, synthetic peptides, aptamers, DARPins or combinations thereof.

This design allows for rapid and custom modification of the nanoparticles with various targets to easily create new panels as wished for by the end user.

According to some embodiments, the coating furthermore comprises a repulsive component provided by a functional group Y attached via a linker to the nanoparticle, wherein functional group Y is chosen from a charged group with a positive or negative charge, a zwitterionic group the method comprises both a positive or negative charge, or a sterically repulsive functional group such as polymer chain or an aliphatic chain.

In an embodiment, the functional group Y may be selected from the group consisting of thiols, disulfides, carboxylic acids, amines, ammonium cations, phosphonic acids, silanes, organosilanes, sulfonates, phosphines, hydroxyls, catechols, gallols, ethoxysilanes, methoxysilanes, silazanes, chlorosilanes, aldehydes, azides, alkynes, cyclooctines (e.g., (Dibenzocyclooctyne (DBCO), trans-cyclooctene (TCO)) Cyclononyne (bicyclo[6.1.0]nonyne (BCN))), tetrazines, avidins, streptavidins, neutravidins and biotins or combinations of avidins/biotins.

In another embodiment the coating comprises a sterically repulsive group, which is also equivalent to functional group Y, such as polymer chain or an aliphatic chain. By the term “sterically repulsive” this refers to groups on the surface of the nanoparticles that through steric hindrance prevent the nanoparticles from agglomerating when in solution.

In an embodiment the polymer may be a water-soluble polymer, such as poly(ethylene glycol)/poly(ethylene oxide), poly(propylene glycol), polypeptide, polyglycerol and polyoxazolines, and combinations thereof. It is preferable that the polymers are of a molecular weight that provides stability to the nanoparticles in solution through steric stability, but still allows for the coating that provides binding affinity of the nanoparticle to the type-specific detection target.

The polymer may be a functional hydrocarbon, optionally selected from the list consisting of polyacrylamide, poly(acrylic acid), poly(methyl methacrylate and poly (methyl acrylate), and combinations thereof.

In another embodiment the functional group Y is a C₂ to C₁₈, such as a C₆ to C₁₈. For example, the aliphatic chain may be selected from the group consisting of hexane, decane, pentadecane, octadecane, polyacetylene, polystyrene and polyethylene, and combinations thereof.

In an embodiment that polymer and/or aliphatic chain has a molecular weight of less than about 4000 Da, such as from about 150 to 4000 Da, such as from about 150 to 2000 Da, for example from about 150 to 1000 Da.

In another embodiment, the nanoparticle coating may comprise a plurality of linkers with conjugating groups, which are configured to conjugate to detection probe X and/or functional group Y.

The conjugating groups may be selected from the list consisting of azides, isothiocyanates, isocyanate, sulfonyl chlorides, aldehydes, carbodiimides, acyl azides, anhydrides, fluorobenzens, carbonates, NHS esters, imidoesters, epoxides, fluorophenyl esters, phosphines, carboxylic acids, maleimides, Haloacetyls (Br—/I—), pyridyl disulfides, thiosulfonates, vinylsulfones alkoxyamines and hydrazides, and combinations thereof.

In an embodiment the nanoparticles in step a. of the method of the invention are provided in solution. Alternatively the nanoparticles may be provided in a dry, solid, form for dispersion in solution prior to contacting with the sample.

Thus, the carefully tailored surface coating of the nanoparticles may comprise a repulsive part and an attractive part (with detection probe) such that they repulse each other and other surfaces to form a stable dispersion while still being able to form specific attractive bonds with a detection target (DNA/RNA or antibodies) resulting in that the nanoparticles attaching and immobilizing on the detection target. Efficient attachment happens when the repulsive forces are balanced with the attractive forces of the probe.

According to some embodiments, the linker comprises an anchor group which tethers the coating to the nanoparticle and optionally a spacer group.

An anchor group “tethering” the coating to the nanoparticle is to be interpreted as that the anchor group binds the coating to the nanoparticle by covalent or non-covalent binding.

According to some embodiments, one or more linkers can provide one or more detection probes X and/or functional groups Y, or where multiple linkers can facilitate provide multiple detection probes and/or functional groups Y via an interconnecting backbone.

Hereby, a plurality of combinations of linkers and detection probes X and/or functional groups Y may be used.

In an embodiment the one or more fluorophores may be selected from the list provided in Table 3 herein.

According to some embodiments, the one or more fluorophores are selected from organic fluorophores, selected from the list consisting of BODIPY, Brilliant Violet, Cyanine, Alexa, Atto, fluorescin, coumarin, rhodamine, xanthene fluorophore families and derivatives, and combinations thereof.

For example the one or more fluorophores are selected from organic fluorophores, selected from the list consisting of Atto 425, Alex fluor 405, Alexa Fluor 488, fluorescin, DiO, Atto 488, Cy3, DiI, Alexa fluor 546, Atto 550, Cy5, Alexa fluor 647, Texas red, DiD, Atto647(N), Atto 655, Cy7, Alexa fluor 680, Alexa fluor 750, Atto 680, and Atto 700, BODIPY, Brilliant Violet, Cyanine, Alexa, Atto, fluorescin, coumarin, rhodamine, xanthene fluorophore families and derivatives and combinations thereof.

According to some embodiments, the plurality of fluorophores are selected from organic fluorophores, selected from the list consisting of Atto 425, Cy3, Cy5 and Cy7, or combinations thereof or from different colors of inorganic fluorophores, wherein the inorganic fluorophores comprises, or are selected from the list consisting of quantum dots, rods, perovskite quantum dots or metal-ligand complexes.

The quantum dots may be semiconductor quantum dots, such as quantum dots that are alloys containing elements from groups III and V of the periodic table, or groups II and VI of the periodic table. Furthermore, the quantum dots may be silicon quantum dots.

By the term “rods” we refer to herein elongated particles where length/width ratio is not equal to 1.

By using well separated fluorophores with high photostability it is possible to achieve a high quality incorporation of these fluorophores in the nanoparticle while minimizing spectral overlap. Both factors are important in order to achieve well separated intensity distributions acting as unique barcode identities (i.e. optical encodings) for each batch of nanoparticles (see FIGS. 4,5,6).

According to some embodiments, the plurality of fluorophores are selected from a combination of different colors of organic fluorophores and different colors of inorganic fluorophores, wherein the inorganic fluorophores comprises quantum dots, rods or perovskite quantum dots.

By using nanoparticles as probes the possibility to combine a broader selection of fluorophores is permitted, especially for the case of semiconducting fluorophores such as quantum dots as mentioned above. This is important because the number of possible combinations scales greater with the number of colors used compared to the number of levels (distinguishable dye concentrations) with the formula n^{m}, where n is the number of intensity levels and m is the number of colors. Using semiconductor fluorophores it is possible to achieve both a narrower emission spectrum (enabling more colors in parallel before spectral overlap becomes a problem), as well as fitting in more colors in a spectrum together with organic fluorophores due to the possibility of having semiconductor fluorophores with very large stoke shifts.

In an embodiment the plurality of nanoparticles are silica nanoparticles, semiconductor nanoparticles, organic nanoparticles, inorganic nanoparticles, metal nanoparticles or polymeric nanoparticles, or combinations thereof.

In another embodiment the plurality of nanoparticles have an average diameter of less than 1000 nm, for example less than 500 nm or 300 nm, or less than 100 nm such as from 3 to 300 nm, for example, from 3 to 200 nm, 3 to 150 nm, or 3 to 100 nm.

The nanoparticle size may be measured by any method known in the art, such as transmission electron microscopy (TEM), scattering electron microscopy (SEM) size exclusion chromatography (SEC) or dynamic light scattering (DLS).

In another embodiment the plurality of nanoparticles are porous nanoparticles, such as mesoporous nanoparticles. For example, the plurality of nanoparticles may be mesoporous silica nanoparticles.

In another embodiment, the plurality of nanoparticles are nanoparticles that have the fluorophore(s) embedded within their matrix.

According to some embodiments, the nanoparticle has at least one of the following characteristics: i. it is a spherical particle comprising of silica and/or semiconductor, organic, inorganic, metal and/or polymer material; ii. it has a diameter of less than 300 nm, preferably less than 200 nm, and more preferably less than 100 nm, even more preferably less than 50 nm.

Spherical silica particles are known from the field, and are an alternative for use in the present invention. Also, nanoparticles of polymers and/or plastics may be used.

Using a particle with a size smaller than about 300 nm, and preferably less than about 100 nm ensures for penetration into cells and that the method can be performed in situ. For the particle to bind to the detection target (after having entered the cell), a smaller size is typically advantageous.

To improve the accessibility of the nanoparticles to the target biomolecules in tissue samples and/or single cells, the latter can be locally permeabilized to allow the target biomolecules to locally diffuse to a 2D or 3D solid phase as defined herein, where they are immobilized via physisorption, electrostatic, hybridization or affinity interactions. In this way, the target biomolecules are fully accessible without the tissue and/or cell matrix while spatial information can still be retrieved.

According to some embodiments, before step c the method of the invention may comprise a step of preparing the target biomolecules for binding with the nanoparticles, such as binding the target biomolecules with at least one molecule comprising the at least one detection target, and/or amplifying the detection targets in situ. Optionally in this step, the target biomolecule is prepared by binding to it at least one molecule such as a barcoded nucleic acid, padlock probe or initiator sequence for subsequent amplification.

This enables the NP probes to be used in assays where enzymatic amplification is omitted, such as regular in situ hybridization (ISH) methods where at least one, but preferably multiple detection targets are bound to a biomolecule in order to generate a stronger signal by binding multiple NPs to a biomolecule.

According to some embodiments, the detection target comprises a nucleic acid molecule, which is, or facilitates a molecule that is, amplified using RCA or multiple hybridization events.

Hence, the signal detection becomes easier and more robust due to (i) a higher signal intensity (ii) the necessity of multiple binding events to generate said signal to avoid randomly immobilized nanoparticles and (iii) utilizing the specificity of molecular tools such as padlock probes for the amplification of the target, avoiding non-targets to be amplified and therefore detected. Similarly, other amplification methods utilizing the binding of for example two or more detection targets in close proximity to allow subsequent hybridizations to generate a stronger but specific signal can be achieved.

According to some embodiments, the decoding is effected by optical decoding such as by optical imaging/fluorescent imaging.

Alternatively, the decoding can be performed in a flow-cytometry-type apparatus that allows the sample to flow through a narrow nozzle with dimensions comparable to a discrete element presenting a specific target biomolecule. The “discrete element” as described herein can be a single cell or a microparticle. The said microparticle can have a probe according to the definition herein to specifically immobilize the target molecule upon presentation to the coded nanoparticles.

This enables high resolution spatial information to be collected with the throughput and resolution of the imaging system and can enable large areas to be scanned.

According to some embodiments, the target biomolecule is further co-labelled with small molecule dyes/probes together with the nanoparticles.

According to some embodiments, further providing one or more molecular probes, each molecular probe comprises a fluorophore that is bound to a nucleic acid molecule, an antigen or an antibody providing binding affinity of the molecular probe to the specific detection target.

As used herein, the term “molecular probe(s)” is referred to interchangeably as “detection oligo(s)”.

The fluorophore of the molecular probe may be any fluorophore, such as any fluorophore detailed herein.

This further improves both robustness and multiplexing where signal detection will rely on both nanoparticle binding event as well as molecular probe binding events to ensure that non-specifically bound nanoparticles are filtered from data analysis. For example, colocalization analysis of nanoprobe signal and detection oligo signal, allowing the removal of false positives from either nanoprobe signal only and detection oligo signal only. This is useful both in-situ and ex-situ, both in tissue and outside tissue. In addition, this can further be utilized to increase the multiplexing capacity by introducing different color of fluorophores acting as molecular probes.

According to a second aspect, the invention relates to a kit-of-parts, comprising, in separate containers:
(i) a plurality of nanoparticles types comprising a plurality of nanoparticles, wherein each nanoparticle comprises a plurality of fluorophores that generates a signal which is unique for each nanoparticle type,
(ii) a probing buffer, comprising a solution with controlled pH, salt concentration and additives facilitating specific detection target binding of the nanoparticle(s); and
(iii) instructions for use of the kit in the method according to the method of the invention.

In an embodiment, the nanoparticles of each nanoparticle type comprise a coating that is configured to conjugate to one or more detection probe X and/or one or more functional group Y.

The detection probe X and functional group Y may be any probe or group as detailed herein in respect of the method of the invention.

In another embodiment, the nanoparticle coating may comprise a plurality of linkers with conjugating groups, which are configured to conjugate to detection probe X and/or functional group Y.

In another embodiment, the nanoparticle coating may comprise a plurality of linkers with conjugating groups, which are conjugated to detection probe X and/or functional group Y.

In another embodiment, each nanoparticle type has a coating that provides binding affinity of the nanoparticle to a specific detection target.

In a particular embodiment of the second aspect of the invention, the kit-of-parts comprises, in separate containers:
(i) nanoparticle(s) of one or more types in a suspension, each nanoparticle type having a coating that provides binding affinity of the nanoparticle to a specific detection target, and wherein each nanoparticle comprises one or more fluorophores that generates a signal which is unique for each nanoparticle type, optionally nanoparticle(s) of each type in a suspensions, each nanoparticle type having a coating without binding affinity of the nanoparticle to a specific detection target wherein the binding affinity is provided in a subsequent step by a detection probe X attached via a linker to the nanoparticle, wherein the detection probe X is chosen from a nucleic acid molecule, an antigen or an antibody;
(ii) optionally, ingredients for providing the one or more nanoparticle type(s) with binding affinity to a specific detection target, comprising a reaction buffer facilitating the binding of detection probe X to the linker, a washing buffer, and a suspension buffer to suspend the nanoparticles in after the introduction of the binding affinity to the coating;
(iii) a probing buffer, comprising a solution with controlled pH, salt concentration and additives facilitating specific detection target binding of the nanoparticle(s); and
(iv) instructions for use of the kit in the method according to the first aspect of the invention.

For the avoidance of doubt, the nanoparticle type(s) of the second aspect of the invention may comprise any of the features of the nanoparticle type(s) of the first aspect of the invention.

In a third aspect of the invention there is provided a nanoparticle comprising a plurality of fluorophores, wherein the nanoparticle is coated with at least one conjugating group which is configured for conjugation to a detection probe.

The nanoparticle may have any of the features of the nanoparticles for any of the “nanoparticle types” disclosed herein in respect of the first and second aspects of the invention.

Thus, among the advantages and unexpected effects of the present invention, the following may also be disclosed:
Time can be saved, experimentally and imaging wise, due to one step vs. multiple for sequencing.
Costs can be saved due to fewer steps.
The method of the present invention is less sensitive to handling errors such as sample integrity and other risks of failure during sequencing procedures.
The present invention allows for an easier data analysis due to one set of images instead of multiple which requires aligning.
The present invention can be used with simple hardware setup by a regularly trained lab person.
The present invention does not require fluidic solutions for iterative chemistry/imaging steps.
The present invention may lower the barrier for adoption of in-situ methods in regular laboratories that lack advanced facilities and instrumentation.
The present invention may be used for multiple applications where a localized signal needs detection/identification.

The present disclosure will become apparent from the detailed description given below. The detailed description and specific examples disclose preferred embodiments of the disclosure by way of illustration only. Those skilled in the art understand from guidance in the detailed description that changes and modifications may be made within the scope of the disclosure. Hence, it is to be understood that the herein disclosed disclosure is not limited to the particular component parts of the device described or steps of the methods described since such device and method may vary. It is also to be understood that the terminology used herein is for purpose of describing particular embodiments only, and is not intended to be limiting. It should be noted that, as used in the specification and the appended claim, the articles “a”, “an”, “the”, and “said” are intended to mean that there are one or more of the elements unless the context explicitly dictates otherwise. Thus, for example, reference to “a unit” or “the unit” may include several devices, and the like. Furthermore, the words “comprising”, “including”, “containing” and similar wordings does not exclude other elements or steps.

For the avoidance of doubt, unless otherwise specified it is intended that any embodiments described above may be couple with the embodiments of the detailed description below if reasonably plausible.

### BRIEF DESCRIPTION OF THE DRAWINGS

The above objects, as well as additional objects, features and advantages of the present disclosure, will be more fully appreciated by reference to the following illustrative and non-limiting detailed description of example embodiments of the present disclosure, when taken in conjunction with the accompanying drawings.

FIG. 1 shows the principle of the invention, wherein (a) shows the situation where one or more nanoparticles (NP) has binding affinity to bind to a specific detection target in a target biomolecule (rolling circle product (RCP)), and (b) shows the situation where the nanoparticle lack binding affinity to the target biomolecule (i.e. no detection target specific for the nanoparticle is present in the biomolecule).

FIG. 2 shows a principal scheme for the preparation of the target biomolecule of the present invention (“Target preparation IP”).

FIG. 3 shows the principles for the coating of the nanoparticles in accordance with the method of the invention (“Coating details for IP”).

FIG. 4 shows the principle of optical encoding/decoding with three nanoparticle types with each a unique optical encoding based on ratiometric control of wavelength and intensity. Spheres represent nanoparticle type with fluorophore(s) incorporated denoted A-C inside the sphere. Coating is established on the nanoparticle surface wherein binding affinity is provided by the detection probe A-C. Decoding is effected by measuring the wavelength and intensity emitted by the nanoparticles, and a ratiometric decoding is illustrated for nanoparticle type A, B and C respectively (1:3:3), (1:0:2) and (3:3:0).

FIG. 5 shows the overlaid emission spectrum of three wavelengths (fluorophores) and three intensity levels.

FIG. 6 is a simulated ratiometric XY-plot of intensity distributions with size and intensity distribution imperfections for a multiplexing system with a population of n=30 with three wavelengths (fluorophores Cy3/Cy5/Cy7) and three intensity levels (relative 0-1-2).

FIG. 7 is a simulated 3D-plot showing the average ratiometric values of each nanoparticle type population for a 26-plex system with three wavelengths (fluorophores Cy3/Cy5/Cy7) and three intensity levels (relative 0-1-2).

FIG. 8 discloses fluorescence intensities measured for 11 different NP type batches produced with precise control of dye incorporation showing the realization of optical encoding of the nanoparticles.

FIG. 9 discloses NP binding (A) specifically to complementary detection targets on biomolecules (RCPs) and (B) not binding to non-complementary targets showing the specificity of the probing properties of the nanoparticle coating.

FIG. 10 (A), shows 2-color encoded NP bound to biomolecules (RCPs) together (i.e. co-labelled) with a molecular probe. (B), A line profile shows the signal emitted in respective channels (Cy3 & Atto 425 from NP and Cy7 from molecular probe). (C) Shows the respective channels for one RCP imaged. (D), shows an XY-plot of the intensity distribution of the decoded NP signal from several RCPs signifying a particular nanoparticle type.

FIG. 11 shows binding of NPs in cells to amplified biomolecules (RCPs) showing the action of in-situ detection of biomolecules using NPs.

FIG. 12 shows a particular embodiment of a coated silica nanoparticle.

FIG. 13 shows the absolute emissions of a 7-plex system according to the invention.

FIG. 14 shows an emission map of a 7-plex system according to the invention.

FIG. 15 shows optical images and corresponding diagrams of coated vs. uncoated nanoparticles in solution.

FIG. 16 shows interbatch titration of Cy3 and Cy5 fluorophore incorporation in the 7-plex system.

FIG. 17 shows 2-plex encoding using detection oligos (molecular probes), where targets 1-7 are distinguished with code 1 (AF750) and targets 8-14 code 2 (Atto425).

### DEFINITIONS

The term “binding” with a target biomolecule, a nanoparticle and/or a detection target is to be interpreted as including the alternative of “hybridization” of nucleic acid molecules to each other.

The term “optical encoding” is to be interpreted as giving a particle a unique signal by incorporating a combination of multiple wavelengths and intensities of fluorophores. For example, the unique signal may be achieved by incorporating predetermined amounts of at least one fluorophore into and/or onto the nanoparticle, thereby arriving at a nanoparticle type that exhibits a specific emission wavelength and intensity.

For the avoidance of doubt, the optical encoding may be achieved by the incorporation of one, or a plurality, of fluorophores into the nanoparticles.

The term “nanoparticle type” is to be interpreted as a nanoparticle having a coating that provides binding affinity to a specific detection target and a unique optical encoding. A plurality of (e.g. at least two) nanoparticle types may be used in accordance with the invention, the various types having binding affinity for different targets. Each nanoparticle type will be represented by a plurality of nanoparticles having the same coating and optical encoding.

The term “nanoparticle” as used herein is also referred to, interchangeably, as the abbreviation “NP”.

### DETAILED DESCRIPTION

Certain aspects of the present disclosure will now be described in further detail. The disclosure may, however, be embodied in other forms and should not be construed as limited to the herein disclosed embodiments. The disclosed embodiments are provided to fully convey the scope of the disclosure to the skilled person.

The first aspect of the invention provides a method for multiplexed detection of a plurality of target biomolecules using optical encoding, wherein each target biomolecule has at least one detection target, comprising the steps of:
a. providing a plurality of nanoparticle types comprising a plurality of nanoparticles, each nanoparticle having a coating that provides binding affinity of the nanoparticle to a type-specific detection target, and wherein each nanoparticle comprises a plurality of fluorophores that generates a signal which is unique for each nanoparticle type;
b. providing a sample comprising a plurality of target biomolecules;
c. contacting the sample with the plurality of nanoparticle types, thereby allowing the nanoparticles to bind with the detection targets of the target biomolecules; and
d. optically decoding the fluorophore signals emitted by the nanoparticle of the nanoparticle type bound to the detection target of the target biomolecules by measuring the wavelength and intensity of the emitted signals, thereby detecting the presence and identity of the target biomolecules.

In another aspect of the invention there is provided a method for multiplexed detection of one or more target biomolecules in situ, wherein each target biomolecule has at least one detection target, using optical encoding, may comprise the steps of: a. providing one or more nanoparticles in a suspension, each nanoparticle having a coating that provides binding affinity of the nanoparticle to a specific detection target, and wherein each nanoparticle comprises one or more fluorophores that generates a signal which is unique for each nanoparticle; b. providing one or more cells to be penetrated by the nanoparticles, wherein the cells comprises one or more target biomolecules; c. optionally preparing the target biomolecules for binding with the nanoparticles, such as binding the target biomolecules with a molecule comprising the detection target, and/or amplifying the detection target in situ; d. contacting the cells with the suspension comprising the nanoparticles, thereby allowing the nanoparticles to penetrate the cells in order to bind with the detection target of the target biomolecule; e. optically decoding the fluorophore signals emitted by the fluorophore of the nanoparticle hybridized to the detection target of the target biomolecules by measuring the wavelength and intensity of the emitted signals, thereby detecting the presence of the target biomolecules.

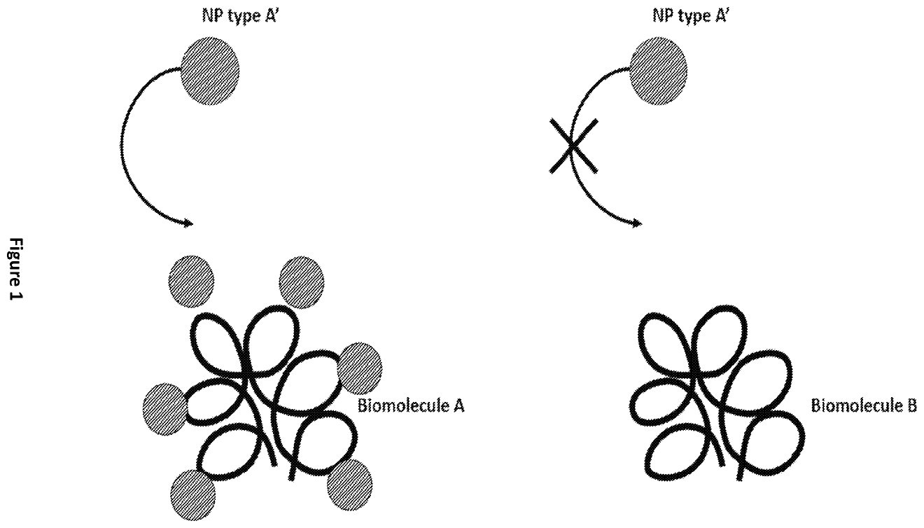

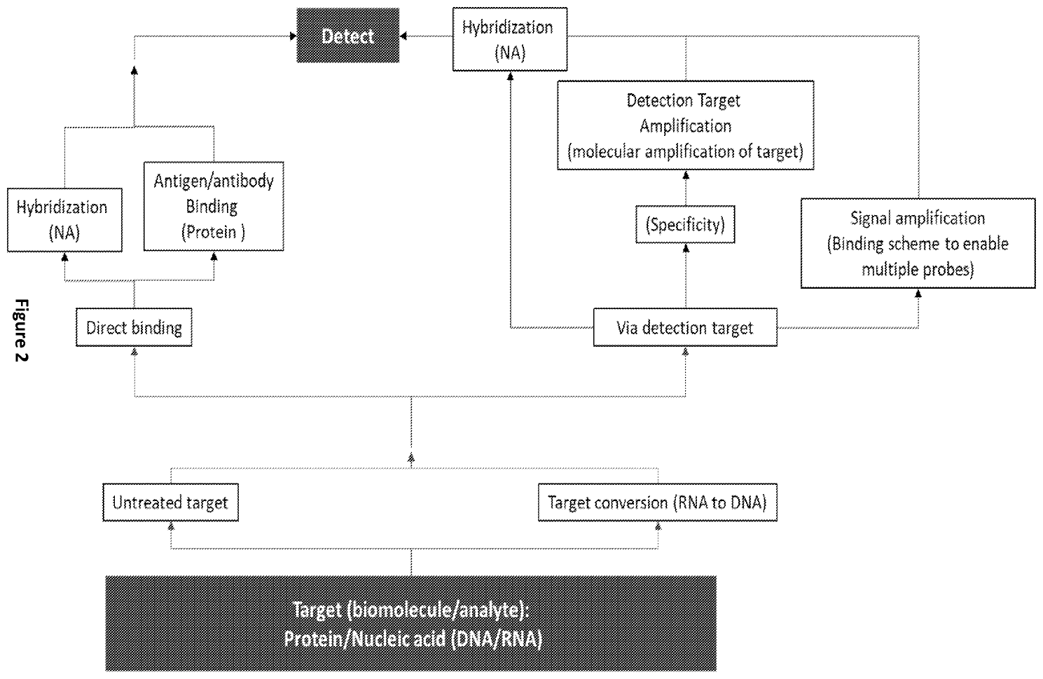

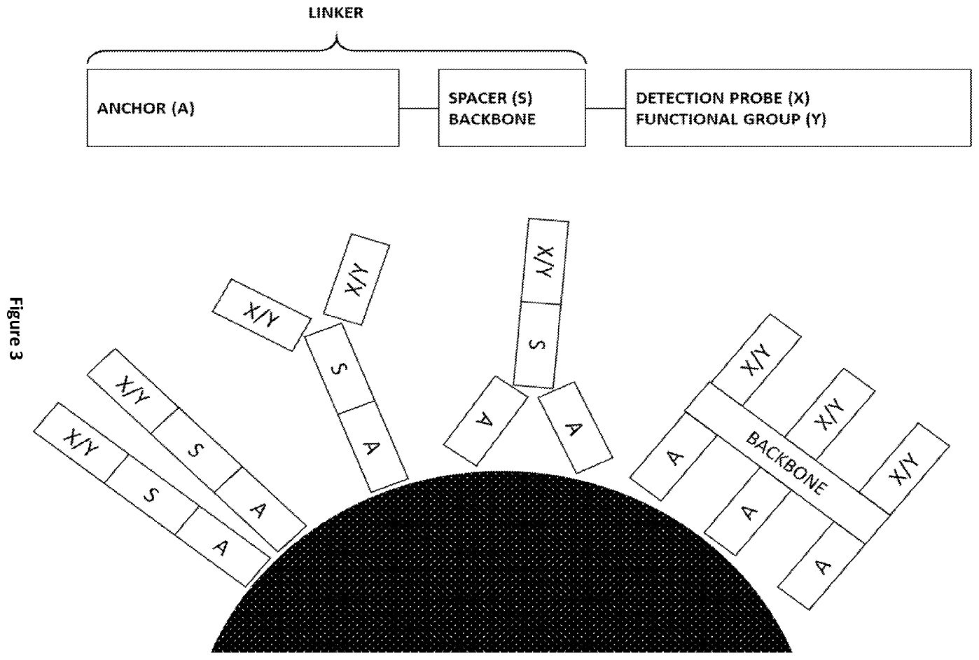

FIG. 1 shows the principles of the present method. FIG. 2 shows the principles for preparing a target for multiplexed detection.

Coating

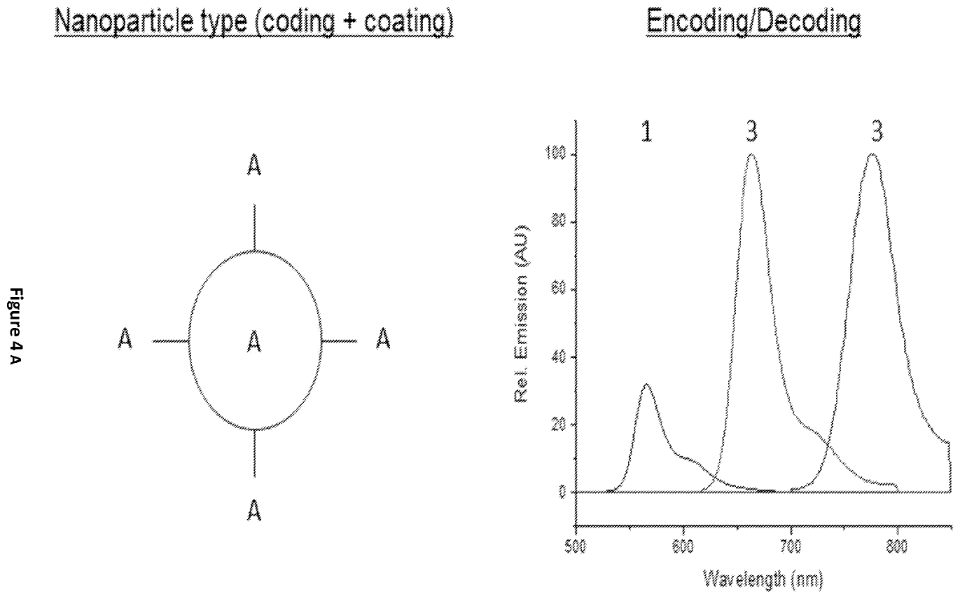

FIG. 3 shows some alternatives for the coating of the nanoparticles of the invention.

The binding affinity of the coating may be provided by a detection probe X attached via a linker to the nanoparticle, wherein detection probe X is chosen from a nucleic acid molecule, an antigen or an antibody.

The coating may furthermore comprise a repulsive component provided by a functional group Y attached via a linker to the nanoparticle, wherein functional group Y is chosen from a charged group with a positive or negative charge, a zwitterionic group the method comprises both a positive or negative charge, or a sterically repulsive functional group such as polymer chain or an aliphatic chain.

Also, the linker may comprise an anchor group A which tethers the coating to the nanoparticle and optionally a spacer group S.

See table 1 for examples of anchor groups and functional groups Y, depending on the surfaces of the nanoparticle. See table 2 for examples of spacer groups. Any of these groups may be used in the context of any aspect of the invention described herein.

TABLE 1

| | Anchor & Functional | Compatible |
|---|---|---|
| Surface | groups | Anchor & Functional groups |
| Metal (Au) | Thiol, Disulfide | |
| | Carboxylic acid | |
| | Amine | |
| | Ammonium cation | |
| | Phosphonic acid | |
| | Silane | |
| | Organosilane | |
| | Sulfonate | |
| | Phosphine | |
| | Hydroxyl | |
| | Catechol | |
| | Gallol | |
| Silica | Ethoxysilane | |
| | Methoxysilane | |
| | Silazane | |
| | Chlorosilane | |
| Functional group | Amine | Isothiocyanate |
| mediated | | Isocyanate |
| | | Sulfonyl Chloride |
| | | Aldehyde |
| | | Carbodiimide |
| | | Acyl azide |
| | | Anhydride |
| | | Fluorobenzen |
| | | Carbonate |
| | | NHS ester |
| | | Imidoester |
| | | Epoxide |
| | | Flurophenyl ester |
| | | Phosphine |
| | | Carboxylic acid |
| | Thiol | Maleimide |
| | | Haloacetyl (Br—/I—) |
| | | Pyridyl disulfide |
| | | Thiosulfonate |
| | | Vinylsulfone |
| | Aldehylde | Alkoxyamine |
| | | Hydrazide |
| Click | Azide | |
| | Alkyne | |
| | Cyclooctines | |
| | (Dibenzocyclooctyne | |
| | (DBCO), trans- | |
| | cyclooctene (TCO)) | |
| | Cyclononyne | |
| | (bicyclo[6.1.0]nonyne | |
| | (BCN)) | |
| | Tetrazine | |
| Ligand | Avidin | |
| | Streptavidin | |
| | NeutrAvidin | |
| | Biotin | |

TABLE 2

| Spacer type | Poly- or Oligo- |
|---|---|
| Bioinert polymers | Poly(ethylene oxide/poly(ethylene glycol), |
| | polypeptide, polyglycerol, polyoxazolines |
| Nucleotide oligomers/ | Non-specific or repeat oligonucleotide |
| polymers | sequences |
| Hydrocarbons | Hexane, decane, pentadecane, octadecane, |
| | polyacetylene, polystyrene, polyethylene, |
| Functional hydrocarbons | Polyacrylamide, poly(acrylic acid), |
| | poly(methyl methacrylate), Poly(methyl |
| | acrylate) |

One or multiple linkers may facilitate one or multiple detection probes X and/or functional groups Y, or where multiple linkers can facilitate multiple detection probes and/or functional groups Y via an interconnecting backbone. For example a polymer chain with functional groups incorporated in the monomer such that multiple anchor and functional groups for linking to detection probe is incorporated in the chain, allowing the chain to orient and bind to the nanoparticle surface using its anchor groups and further be modified with detection probes using linkers that form bonds with the remaining functional groups.

Fluorophores

One or more fluorophores may be selected from the alternatives provided in table 3 and derivatives thereof.

TABLE 3

| Fluorophore | |
|---|---|
| Type | Fluorophore Name |
| Metallorganic | |
| Semiconductor | Semiconductor quantum Dots (III-V, II-VI, Si) |
| | Perovskite quantum dots |
| | Carbon quantum dots |
| | Quantum rods |
| | P-dots (e.g. organic chromophoric polymers, whose surfaces can be |
| | modified with different amphiphilic polymers) |
| | Silicon quantum dots |
| Blue | Atto 425 |
| | 4-[3-(ethoxycarbonyl)-6,8,8-trimethyl-2-oxo-7,8-dihydro-2H- |
| | pyrano[3,2-g]quinolin-9(6H)-yl]butanoic acid |
| | Alexa fluor 405 |
| | tris(N,N-diethylethanaminium) 8-[2-(4-{[(2,5-dioxopyrrolidin-1- |
| | yl)oxy]carbonyl}piperidin-1-yl)-2-oxoethoxy]pyrene-1,3,6- |
| | trisulfonate |
| Green | Alexa fluor 488 |
| | 6-amino-9-(2,4-dicarboxyphenyl)-4,5-disulfo-3H-xanthen-3-iminium |
| | Fluorescin |
| | 3′,6′-dihydroxyspiro[isobenzofuran-1(3H),9′-[9H]xanthen]-3-one |
| | DiO |
| | 3,3′-Dioctadecyloxacarbocyanine Perchlorate |
| | Atto 488 |
| | BODIPY FL |
| | 3-{2-[(3,5-Dimethyl-1H-pyrrol-2-yl-κN)methylene]-2H-pyrrol-5-yl- |
| | κN}propanoato)(difluoro)boron |
| Yellow - Orange | Cy3 (amine and derivates) |
| | 6-[6-[(2E)-3,3-dimethyl-2-[(E)-3-(1,3,3-trirnethylindol-1-ium-2- |
| | yl)prop-2-enylidene]indol-1- |
| | yl]hexanoylamino]hexylazanium; dichloride |
| | DiI |
| | 1,1′-Dioctadecyl-3,3,3′,3′-Tetramethylindocarbocyanine |
| | Alexa fluor 546 |
| | 2,3,5-trichloro-4-{[({6-[(2,5-dioxopyrrolidin-1- |
| | yl)oxy]-6-oxohexyl}carbamoyl)methyl]sulfanyl}-6- |
| | (2,2,4,8,10,10-hexamethyl-12,14-disulfo- |
| | 2,3,4,8,9,10-hexahydro-1H-13-oxa-1,11- |
| | diazapentacen-6-yl)benzoic acid |
| | Atto 550 |
| | N/A |
| | BODIPY TMR-X |
| | [N-{6-[(2,5-dioxopyrrolidin-1-yl)oxy]-6-oxohexyl}-3-(2-{[5-(4- |
| | methoxyphenyl)-1H-pyrrol-2-yl-kappaN]methylene}-3,5-dimethyl-2H- |
| | pyrrol-4-yl-kappaN)propanamidato](difluoro)boron |
| Red | Cy5 |
| | 6-[6-[(2E)-3,3-dimethyl-2-[(2E,4E)-5-(1,3,3-trimethylindol-1-ium- |
| | 2-yl)penta-2,4-dienylidene]indol-1-yl]hexanoylamino]hexylazanium |
| | Alexa fluor 647 |
| | 2-[5-[3,3-dimethyl-5-sulfo-1-(3-sulfopropyl)indol-1-ium-2-yl]penta- |
| | 2,4-dienylidene]-3-methyl-3-[5-oxo-5-(6- |
| | phosphonooxyhexylamino)pentyl]-1-(3-sulfopropyl)indole-5-sulfonic |
| | acid |
| | Texas Red |
| | 5-chlorosulfonyl-2-(3-oxa-23-aza-9- |
| | azoniaheptacyclo[17.7.1.15,9.02,17.04,15.023,27.013,28]octacosa- |
| | 1(27),2(17),4,9(28),13,15,18-heptaen-16-yl)benzenesulfonate |
| | DiD |
| | 1,1′-Dioctadecyl-3,3,3′,3′-Tetramethylindodicarbocyanine |
| | Atto 647(N), Atto 655 |
| | N/A |
| Near-IR | Cy7 |
| | 1-(5-carboxypentyl)-2-[7-(1-ethyl-5-sulfo-1,3-dihydro-2H-indol-2- |
| | ylidene)hepta-1,3,5-trien-1-yl]-3H-indolium-5-sulfonate |
| | Alexa fluor 680 |
| | N/A |
| | Alexa fluor 750 |
| | N/A |
| | Atto 680, Atto 700 |
| | N/A |

Nanoparticle

The nanoparticle may fulfil the following characteristics:
The size of the nanoparticle may be small enough for the nanoparticle to be able to penetrate the cell membrane, i.e. typically a diameter of less than 100 nm is required.
The coating of the nanoparticle may allow specific targeting of the nanoparticle to the detection target in question. Also, it is important and/or advantageous that the coating is such that aggregation is avoided, by tailoring the repulsive forces (for example cationic, anionic, zwitterionic or steric repulsive forces) of the coating and/or modifying the formulation of the buffers used during binding to detection targets.
The plexing capacity of the nanoparticle is important. Preferably more than 50 and up to or more than 100 different codes should be possible to detect. Thus, challenges of quenching, dye stability and level discernment must be solved. By carefully designing the nanoparticle architecture, optionally utilizing the size to further incorporate both organic dyes as well as inorganic dyes, and with the possibility to further modify the matrix and structure of the nanoparticle the stability of dyes can be increased as well as increasing the flexibility of combining various fluorophores that can work in synchronization to generate a high magnitude of optical encoding.
However, for the avoidance of doubt, plexing will be achieved as long as there are at least two nanoparticle types used. For example, plexing may be achieved with at least 3, 4, 5, 6, 7, 8,9 or 10 nanoparticle types.
Guidance for synthetization of nanoparticles for use according to the invention, with a suitable size, coating and fluorophore content, can be found in literature of the art, such as e.g. Wang et al. (Nanoletters, 2005; 5:1 (37-43)).
The examples also provide detailed guidance on how to synthesize nanoparticles for use in the invention.

Target Biomolecule Preparation

In the method of the invention, the target biomolecule may be prepared by binding it to a molecule the method comprises the detection target, such as a barcoded padlock probe or initiator sequence for HCR or other amplification methods.

The detection target may be a nucleic acid molecule or a protein, which is amplified using a method as listed in table 4.

TABLE 4

| Method | Details |
|---|---|
| Rolling circle | Padlock-probe mediated, clonal amplification |
| amplification mediated | method using the concept of rolling circle |
| | amplification. This is the most common in |
| | the emerging in-situ technologies. |
| | The padlock probe can be ligated directly on |
| | RNA transcripts, genomic DNA or on cDNA. |
| | The amplification can be performed using |
| | the target nucleic acid as primer or an |
| | external primer hybridizing to the backbone |
| | of the padlock probe. |
| Multiple | The concept of multiple hybridizations to |
| hybridizations | generate a stronger signal is the common |
| (ISH) mediated: | factor, one can design this in a multitude of |
| HCR (Hairpin chain | different ways where the detection target is |
| reaction), and | being built up using several hybridization |
| probably more | events. This is what's been used |
| | traditionally with FISH. After these |
| | hybridization events, the hybridization |
| | probes/complexes may be further amplified |
| | using rolling circle amplification |
| Transposase-mediated | Cleavage of genomic DNA and generation of |
| amplification | dumbbell structures for rolling circle |
| | amplification. |
| | Insertion of specific sequences for |
| | subsequent amplification such as a T7 RNA |
| | promoter. |
| | After the first round of rolling circle |
| | amplification or transcription, the |
| | amplification products can be |
| | detected/genotyped/sequenced in a second |
| | round of padlock probe mediated |
| | amplification. |
| Antibody or | Probes with specific affinity to proteins in |
| aptamer-mediated | the samples are pre-conjugated with a |
| amplification | specific oligonucleotide sequence serving as |
| | initiation site for an amplification event. |
| | Upon contact with the sample, these probes |
| | recognize specific targets in the sample and |
| | the remaining probes are washed away. The |
| | probes bound to specific targets are used as |
| | initiation sites for rolling circle amplification |
| | and subsequently detected using |
| | nanoparticles according to embodiments |
| | herein. |

Optical Encoding & Decodine

The optical encoding may be effected by incorporating a plurality of fluorophores (e.g. of different wavelengths) in precisely controlled ratios (intensities) such that each combination of wavelengths/intensity ratios becomes uniquely separable when decoding thereby optically encoding each nanoparticle type. The different ratios of intensities can be achieved by (i) controlling the concentration of fluorophores in the nanoparticle and/or (ii) altering the size or optical properties of the fluorophores to control its emission intensity and thereby achieving ratiometric read-out. It is understood that the fluorophores incorporated in the nanoparticle may be encapsulated by its matrix and also placed at the surface of the nanoparticle.

Furthermore, each nanoparticle type is given a unique binding affinity through its coating such that it can recognize a unique detection target.

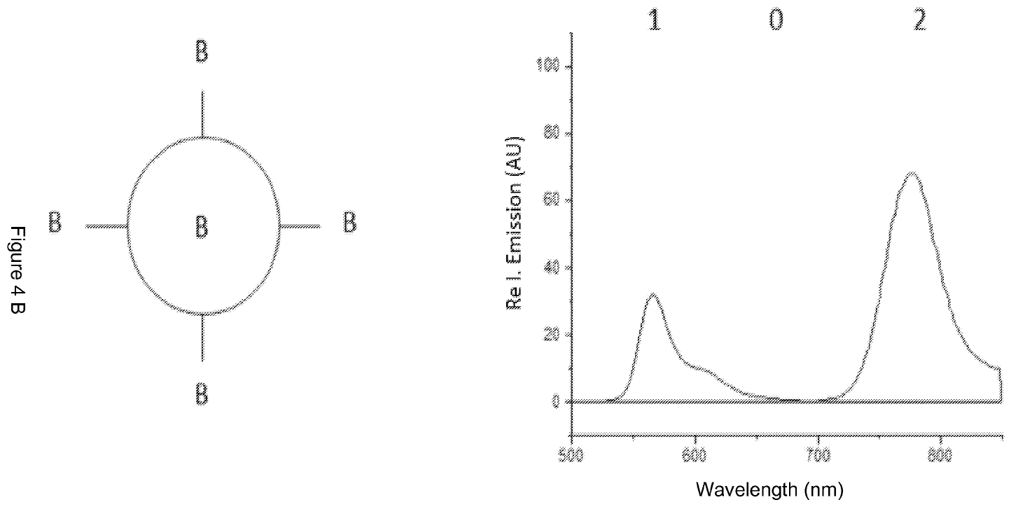

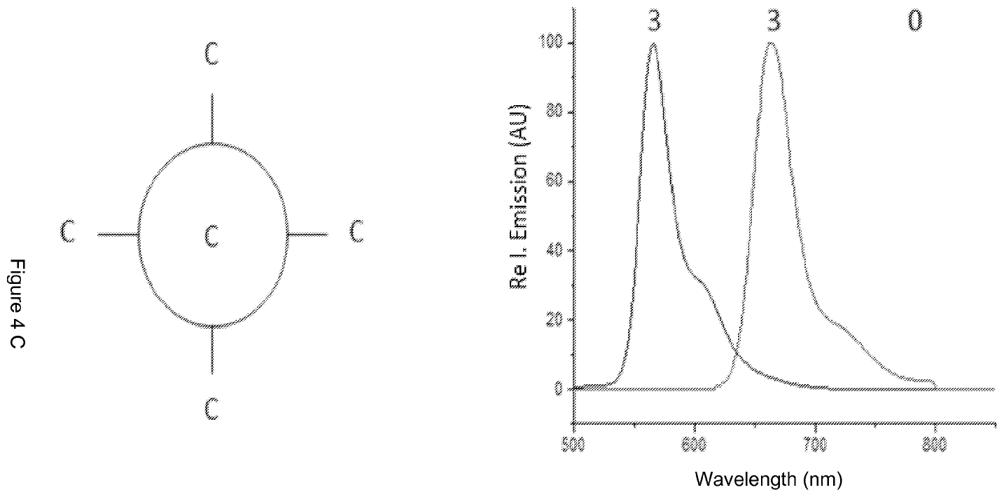

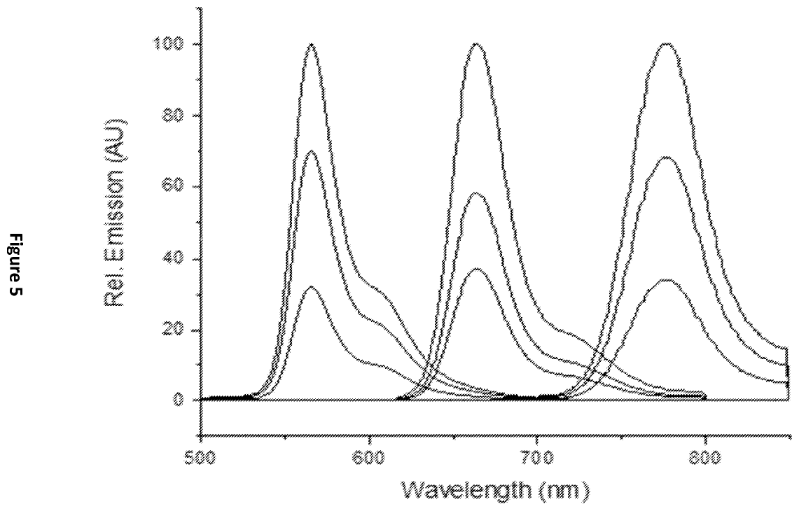

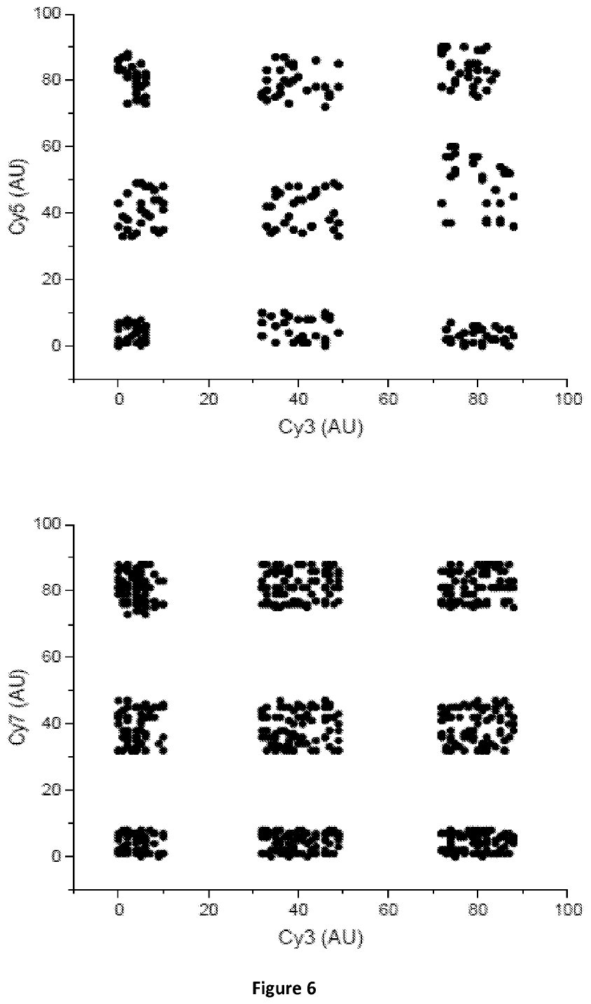

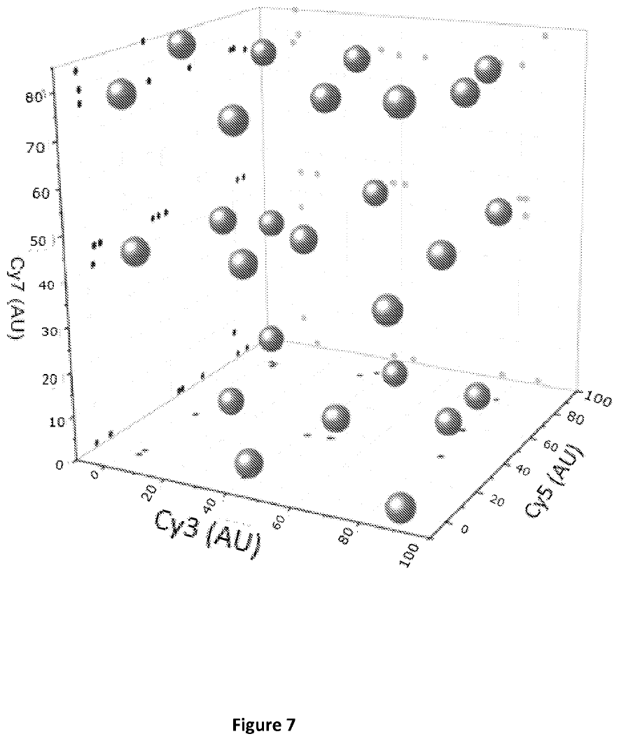

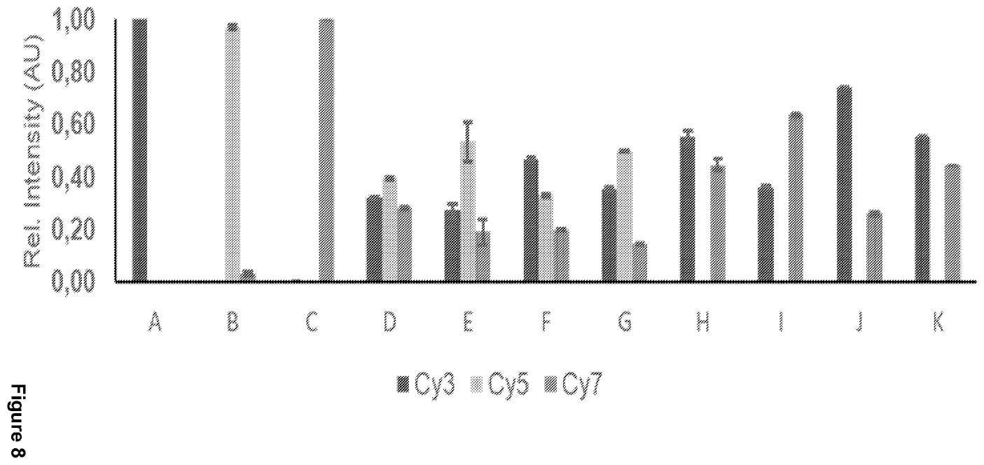

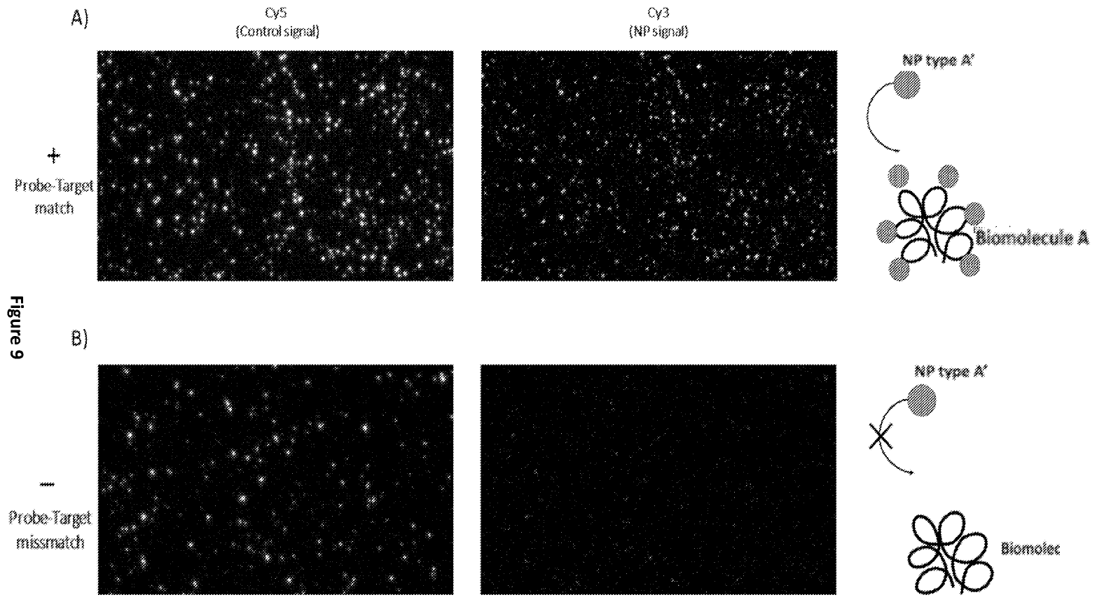

The decoding may be effected by optical decoding such as by optical imaging of the fluorescent signals. The signal from each nanoparticle type may be identified by measuring the wavelengths and intensities in order to ratiometrically identify the optical code incorporated in the nanoparticle types as shown in FIG. 4.

The target biomolecule may be further co-labelled with small molecule probes (comprising their own fluorophores) together with the nanoparticles. This increases the robustness of the assay by introducing a control signal showing where specific binding occurs between nanoparticles and detection target of the biomolecule(s), and furthermore may introduce another dimension in the optical encoding increasing the degree of multiplexity by combining the signal emitted from the nanoparticle with the signal emitted from the biomolecule. Therefore, the identity of the biomolecule can be determined by using optically encoded nanoparticles together with additionally encoding the biomolecule with non-nanoparticle probes, for example by extending the number of wavelengths (fluorophore signals) emitted by the nanoparticle-biomolecule complex by co-labeling with a probe containing a molecular fluorophore.

### Specific Embodiments of the Invention

Specific embodiments of the invention are provided in the paragraphs below.

In an embodiment of the invention there is provided a method for multiplexed detection of one or more target biomolecule(s) in situ using optical encoding, wherein each target biomolecule has at least one detection target, comprising the steps of:
a. providing one or more nanoparticle type(s) in a suspension, each nanoparticle having a coating that provides binding affinity of the nanoparticle to a type-specific detection target, and wherein each nanoparticle comprises one or more fluorophore(s) that generates a signal which is unique for each nanoparticle type;
b. providing one or more cells to be penetrated by the nanoparticle(s), wherein the cell(s) comprise(s) one or more target biomolecule(s);
c. optionally preparing the target biomolecule(s) for binding with the nanoparticle(s), such as binding the target biomolecule(s) with at least one molecule comprising the at least one detection target, and/or amplifying the detection target(s) in situ;
d. contacting the cells with the suspension comprising the nanoparticle(s), thereby allowing the nanoparticle(s) to penetrate the cells in order to bind with the detection target(s) of the target biomolecule;
e. optically decoding the fluorophore signal(s) emitted by the nanoparticle of the nanoparticle type bound to the detection target of the target biomolecule(s) by measuring the wavelength and intensity of the emitted signal(s), thereby detecting the presence and identity of the target biomolecule(s).

Preferably each nanoparticle type is optically encoded by (i) incorporating precisely controlled ratios of at least one fluorophore, thereby controlling the emission wavelength and intensity from the nanoparticle type, or (ii) altering the properties of the fluorophore(s) affecting its emission intensity.

Advantageously the binding affinity of the coating of the nanoparticle type is provided by a detection probe X attached via a linker to the nanoparticle, wherein the detection probe X is chosen from a nucleic acid molecule, an antigen or an antibody.

Conveniently the coating of the nanoparticle furthermore comprises a repulsive component provided by a functional group Y attached via a linker to the nanoparticle, wherein the functional group Y is chosen from a charged group with a positive or negative charge, a zwitterionic group comprising both a positive or negative charge, or a sterically repulsive functional group such as polymer chain or an aliphatic chain.

Preferably the linker comprises at least one anchor group, which tethers the coating to the nanoparticle and optionally a spacer group.

Advantageously one or more linkers can provide one or more detection probes X and/or functional groups Y, or where multiple linkers can provide multiple detection probes X and/or functional groups Y via an interconnecting backbone.

Conveniently the one or more fluorophores are chosen from:
(i) organic fluorophores, chosen from Atto 425, Cy3, Cy5, and Cy7;
(ii) different colors of inorganic fluorophores, wherein the inorganic fluorophores comprises quantum dots, rods, perovskite quantum dots or metal-ligand complexes; and/or
(iii) a combination of different colors of organic fluorophores and different colors of inorganic fluorophores, wherein the inorganic fluorophores comprises quantum dots, rods or perovskite quantum dots.

Preferably the nanoparticle has at least one of the following characteristics:
i. it is a spherical particle comprising of silica and/or semiconductor, organic, inorganic, metal and/or polymer material;
ii. it has a diameter of less than 300 nm, preferably less than 200 nm, and more preferably less than 100 nm, even more preferably less than 50 nm.

Advantageously in step c, the target biomolecule is prepared by binding to it at least one molecule comprising the detection target, such as a barcoded nucleic acid molecule, padlock probe or initiator sequence for subsequent amplification.

Conveniently the detection target comprises a nucleic acid molecule, which is, or facilitates a molecule that is, amplified using RCA or multiple hybridization events.

Preferably the decoding is effected by optical decoding such as by optical imaging.

Advantageously the method further comprises the step of providing one or more molecular probes, wherein each molecular probe comprises a fluorophore that is bound to a nucleic acid molecule, an antigen or an antibody providing binding affinity of the molecular probe to the specific detection target.

In another aspect of the invention there is provided a kit of parts, comprising, in separate containers,
(i) nanoparticle(s) of one or more types in a suspension, each nanoparticle type having a coating that provides binding affinity of the nanoparticle to a specific detection target, and wherein each nanoparticle comprises one or more fluorophores that generates a signal which is unique for each nanoparticle type, optionally nanoparticle(s) of each type in a suspensions, each nanoparticle type having a coating without binding affinity of the nanoparticle to a specific detection target wherein the binding affinity is provided in a subsequent step by a detection probe X attached via a linker to the nanoparticle, wherein the detection probe X is chosen from a nucleic acid molecule, an antigen or an antibody;
(ii) optionally, ingredients for providing the one or more nanoparticle type(s) with binding affinity to a specific detection target, comprising a reaction buffer facilitating the binding of detection probe X to the linker, a washing buffer, and a suspension buffer to suspend the nanoparticles in after the introduction of the binding affinity to the coating;
(iii) a probing buffer, comprising a solution with controlled pH, salt concentration and additives facilitating specific detection target binding of the nanoparticle(s); and
(iv) instructions for use of the kit in the method according to any of the paragraphs above in the section titled “Specific Embodiments of the Invention”.

The person skilled in the art realizes that the present disclosure is not limited to the preferred embodiments described above. The person skilled in the art further realizes that modifications and variations are possible within the scope of the appended claims.

Additionally, variations to the disclosed embodiments can be understood and effected by the skilled person in practicing the claimed disclosure, from a study of the drawings, the disclosure, and the appended claims.

Sequences

SEQ ID No: 1
TTTTTTTTTTCCTCAGTAATAGTGTCTTAC
SEQ ID No: 2
TTACACCCTTTCTTTGACAA GTGTATGCAGCTCCTCAGTAATAGTGTCT
TTGTGCGTCTATTTAGTGGAGCC CATG GACTGTTACTGAGCTGCGTT
SEQ ID No: 3
TTGTCAAAGAAAGGGTGTAAAACGCAGCTCAGTAACAGTC
SEQ ID No: 4
TGCGTCTATTTAGTGGAGCC
SEQ ID No: 5
DBCO Oligo (SARS-COV-2)
5′-DBCO-TTTTTTTTTTTTCCTCAGTAATAGTGTCTTAC-3′
SEQ ID No: 6
DBCO oligo (HIV)
5′-DBCO-TTTTTTTTTTTGCGTCTATTTAGTGGAGCC-3′
SEQ ID No: 7
Synthetic target HIV
5′-
CTCTCTCTCTCTATACTATATGTTTTAGTTTATATTGTTTCTTTCCCCCT
GGCCTTAACCGAATTTTTTCCCATTTATCTAATTCTCCCCCGCT-3′
SEQ ID No: 8
Synthetic target SARS-COV-2
5′-Biotin-
CTCTCTCTCTCTCTCTCTCTCTCTCTCTCTCGGTGTGACAAGCTACAACA
CGTTGTATGTTTGCGAGCAA-3′
SEQ ID No: 9
Padlock Probe HIV
5′-PO₄-AAGGCCAGGGGGAAAG AGTAGCCGTGACTATCGACT TGCGT
CTATTTAGTGGAGCCTTAAATGGGAAAAAATTCGGTT-3′
SEQ ID No: 10
Padlock probe SARS-COV-2
5′-PO₄-
TGTTGTAGCTTGTCACACCGGTGTATGCAGCTCCTCAGTAATAGTGTCTT
ACGGCATCACTGGTTACGTCTGTTGCTCGCAAACATACAACG-3′
SEQ ID No: 11
AF750-Detection oligo (SARS-COV-2)
5′-TCCTCAGTAATAGTGTCTTACTTTT-AF750-3′
SEQ ID No: 12
Atto425-Detection oligo
5′-Atto425-CCTCAGTAATAGTGTCTTAC-3′

### EXAMPLES

### List of Abbreviations

NPs: Nanoparticles
EtOH: Ethanol
PBS: Phosphate buffered saline
DEPC: Diethyl pyrocarbonate treated
RCA: Rolling circle amplification
RCP: Rolling circle product, the result of rolling circle amplification
BSA: Bovine serum albumin
dNTP: Deoxyribonucleotide triphosphate
ATP: Adenosine triphosphate
PEG: Polyethylene glycol
SSC: Saline-sodium citrate
EDTA: Ethylenediaminetetraacetic acid

### Example 1—Suspension Conditions

In order to perform the claimed method, the conditions of the suspension, in which the method takes place, may have the following components and characteristics:

The suspension conditions can be controlled such that specific binding of nanoparticles to detection targets is facilitated over unspecific binding to other targets or non-targets (such as other biological matter/matrix or surface) while maintaining the stability of nanoparticle dispersion.

The following components play important role in optimizing NP binding to target biomolecules.

TABLE 5
Overview of components in probing/hybridization buffer.

| Type (effect) | Examples | | |
|---|---|---|---|
| Denaturants (lower | Formamide/Urea/ethylene | 0-60% | (v/v) |
| hybridization T) | glycol/ethylene | | |
| | carbonate/sodium | | |
| | percholarate etc. | | |
| Salts & Buffer (lower | NaCl, KCl, MgCl₂ | 0-3000 | mM |
| electrostatic repulsion) | Citrate buffer | 0-300 | mM |
| | Phosphate buffer | 0-300 | mM |
| | (Buffers usually used as | | |
| | SSC, PBS) etc. | | |
| pH (charge of nucleic acids) | Adjusted with HCl/NaOH | 6-9 | |
| Nuclease inhibitor | EDTA, Riboprotect nuclease | 0-10 | mM |
| | inhibitor | 0.1-5 | U/μL |
| Detergent (prevent | SDS, Tween, Triton X-100, | 0-5% | (v/v or m/v) |
| nonspecific binding) | N-lauroylsarcosine etc. | | |
| Blocking agents (prevent | BSA, Casein, salmon sperm | 0-10% | (v/v or m/v) |
| nonspecific binding) | DNA, denatured ssDNA etc. | | |

| | | |
|---|---|---|
| Hybridization accelerators, | dextran sulfate, PEG, ficoll | 0-50% (e.g. 0-20%) |
| crowding agents | etc. | (v/v or m/v) |

In one embodiment, the following conditions were used:

| SSC | 2X |
|---|---|
| Formamide | 20% |
| PEG 4000 | 5% |
| pH | 7-7.4 |

In another embodiment, the following conditions were used:

| SSC | 1X |
|---|---|
| Formamide | 10% |
| PEG 4000 | 5% |
| pH | 7-7.4 |

### Example 2—Using the Method for Multiplexed Detection

TABLE 6
Overview of sequences used in examples

| ID | Ref | Sequence |
|---|---|---|
| 1 | S04018 | TTTTTTTTTTCCTCAGTAATAGTGTCTTAC |
| 2 | S03625 | TTACACCCTTTCTTTGACAA GTGTATGCAGCTCCTCAGT |
| | | AATAGTGTCTTTGTGCGTCTATTTAGTGGAGCC CATG |
| | | GACTGTTACTGAGCTGCGTT |
| 3 | S03844 | TTGTCAAAGAAAGGGTGTAAAACGCAGCTCAGTAACAGTC |
| 4 | S03798 | TGCGTCTATTTAGTGGAGCC |

In one embodiment, NP surface coating was performed by adding 100 μL of NPs (125 mM Si, 0.62 uM Cy3 loading) to a solution of H2O (485 μL) followed by 11-azidoundecyltriethoxysilane (2.5 μL, 256 mM), mPEG5k-triethoxysilane (10 μL, 20 mM) and finally ammonium hydroxide (2 μL, 28-30%). The temperature was raised to 75° C. and the reaction mixture was shaken. After 3 hours the reaction mixture was washed by centrifugation 3 times with H₂O and 1 time with PBS. The coated NPs were stored in 100 μL H₂O.

To EtOH (50 μL), above coated NPs were added (30 μL) followed by PBS (20 μL) and DBCO modified nucleotide sequence S04018 (3 μL, 100 uM). The temperature was raised to 37° C. during shaking. After 19 hours the reaction mixture was washed by centrifugation 3 times with H2O. The sequence functionalized NPs were stored in 100 μL H₂O.

In one embodiment, RCPs were prepared in solution by adding 5′-phosphorylated padlock probes S03625 (1 μL, 1 uM) to a solution of H2O (71.9 μL), phi29 reaction buffer (10 μL, 10×), T4 ligase (0.4 μL, 5 U/μL), BSA (10 μL, 2 μg/μL), ATP (2.7 μL, 25 mM) followed by target sequence S03844 (4 μL, 30 nM). The temperature was raised to 37° C. for 30 minutes. From this, 20 μL (first diluted 1:1000) was added to H₂O (9.2 μL) followed by BSA (4 μL, 2 μg/μL), dNTPs (2 μL, 2.5 mM), phi 29 reaction buffer (4 μL, 10×) and phi 29 polymerase (0.8 μL, 10 U/μL). Temperature was raised to 30° C. for 2 hours followed by 65° C. for 5 minutes.

In one embodiment, NPs binding to RCPs were performed by first immobilizing 5 μL of RCPs to Superfrost Plus slides (Thermo Scientific) by deposition. The proceeding reactions were performed in Secure-seals (Grace Bio-Labs, 9 mm in diameter and 0.8 mm deep) attached to the slides. Hybridization mixture was prepared by adding NPs (5 μL) to a solution of SSC (5 μL, 20×), formamide (10 μL), H2O (24.5 μL), PEG 4000 (5 μL, 50%) followed by complementary detection oligo 503798-Cy5 (control probe). To the immobilized RCPs was added the hybridization mixture and temperature was raised to 37° C. After one hour the glass slide was washed 4 times with PBS-T (DEPC-PBS with 0.05% Tween-20), once with 70% EtOH, once with 85% EtOH and finally 99.5% EtOH.

In one embodiment, RCP generation in situ was performed instead of in solution. Cells were seeded on Superfrost Plus slides (Thermo Scientific). When the cells reached the desired confluency they were fixed in paraformaldehyde (3% (w/v)) in PBS at room temperature. After 30 min, slides were washed twice with DEPC-PBS once with 70% EtOH, once with 85% EtOH and finally 99.5% EtOH for 3 min each. The reactions were performed in Secure-seals (Grace Bio-Labs, 9 mm in diameter and 0.8 mm deep) attached to the slides. To make the RNA more readily available for cDNA synthesis, 0.1 M HCl was applied to the cells for 10 min at room temperature. This was followed by two washes in DEPC-PBS.

Reverse transcription was performed by adding a mixture of DEPC-H₂O (34.75 μL), TranscriptMe buffer (5 μL, 10×), dNTPs (1 ML, 25 mM), BSA (0.5 ML, 20 μg/μL), random decamers (2.5 μL, 100 uM), RiboLock RNase Inhibitor (Fermentas) (1.25 μL, 40 U/μL) and reverse transcriptase (5 μL, 200 U/μL) to the cells and temperature was raised to 37° C. After 18 hours, the reaction mixture was removed and formaldehyde (3% (w/v)) was added. After 30 minutes the cells were washed with PBS-T twice.

Ligation was performed by adding a mixture of DEPC-H₂O (22 μL), Ampligase buffer (20 mM Tris-HCl, pH 8.3, 25 mM KCl, 10 mM MgCl₂, 0.5 mM NAD and 0.01% Triton X-100) (5 μL, 10×), KCl (2.5 μL, 1 M), formamide (10 μL), padlock probes (1 μL, 0.5 uM), BSA (0.5 μL, 20 μg/μL), Ampligase (5 μL, 5 U/μL) and RNase H (4 μL, 5 U/μL) to the cells and temperature raised to 37° C. for 30 minutes and then 45° C. After 1 hour, cells were washed with PBS-T twice.

RCA was performed by adding a mixture of DEPC-H₂O (33 μL), Phi-29 buffer (5 μL, 10×), glycerol (5 μL, 50%), dNTPs (0.5 μL, 25 mM), BSA (0.5 μL, 20 μg/μL), Exonuclease I (1 μL, 20 U/μL), Phi-29 polymerase (5 μL, 10 U/μL) to the cells and raising the temperature to 37° C. After 4 hours, cells were washed with PBS-T twice. Subsequent NP and control probe binding was performed as above analogous to RCPs deposited on slides.

In one embodiment, image acquisition was performed using an Axioplan II epifluorescence microscope (Zeiss) equipped with a 100 W mercury lamp, a CCD camera (C4742-95, Hamamatsu), and a computer-controlled filter wheel with excitation and emission filters for visualization of 425, DAPI, FITC, Cy3, Cy3.5, Cy5 and Cy7. A ×20 (Plan-Apocromat, Zeiss), ×40 (Plan-Neofluar, Zeiss) or ×63 (Plan-Neofluar, Zeiss) objective was used for capturing the images.

### Example 3—Nanoparticle Synthesis

Materials

Cyanine 3 NHS ester (Cy3-NHS), Cyanine 5 NHS ester (Cy5-NHS) were purchased from Lumiprobe GmbH, Germany. Dimethyl Sulfoxide (DMSO) (≥99.9%), Tetrahydrofuran (THF (99%) (3-aminopropyl)triethoxysilane (APTES), ammonium hydroxide (NH4OH) (28% NH3 in H2O, ≥99.99%), tetraethyl orthosilicate (TEOS) (99.999%), 11-azidoundecyltriethoxysilane (97%), ADIBO-PEG4-acid (90%) were purchased from Sigma Aldrich, Sweden. Ethanol absolute (≥99.8%) was purchased from VWR, Sweden. Ultra-pure MilliQ water used from MilliQ (IQ 7010) system.

Fluorophore Stock

Stock solutions of fluorophores were prepared by adding 1 ml of DMSO to 1 mg of respective fluorophore.

Optically Encoded Nanoparticles Library

Encoded NPs were synthesized by adding either of [0, 0.8, 1.6, 3.2, 6.4] μl Cy3-NHS, Cy5-NHS, Cy7-NHS respectively to 4 μL of APTES solution and raising the temperature to 37° C. during stirring or shaking. The APTES solution was prepared by first adding 10 μl of APTES to 990 μl of EtOH. For example, to produce a nanoparticle type batch of encoding [8, 16, 0], 0.8 μL of Cy3-NHS, 1.6 μL of Cy5-NHS and 0 μL of Cy7-NHS was added. This way any combination can be made using for example 3 colors and 5 levels according to above example. After 10 minutes, 38 μl of H₂O was added followed by EtOH and the temperature was raised to 55° C. The amount of EtOH was set to reach a final reaction volume of 1 mL after the following additions; 54 μl of NH₄OH was added followed by 38 μl of TEOS during vigorous stirring. After 2 hours, the NPs were washed by centrifugation 3 times using EtOH. The NPs were then redispersed in 1 mL EtOH and stored at 4° C. until further use.

The resulting nanoparticles had an average diameter size of 66 nm as measured by SEM and 75 nm as measured by DLS.

In one embodiment, a 7-plex encoded NP system was synthesized using above method with the following encodings: (32:0, 32:8, 32:16, 32:32, 16:32, 8:32, 0:32).

In another embodiment, a 15-plex encoded NP system was synthesized using above method with the following encodings: (8:0:16, 16:0:16, 32:0:16, 0:8:16. 8:8:16, 16:8:16, 32:8:16, 0:16:16, 8:16:16, 16:16:16, 32:16:16, 0:32:16, 8:32:16, 16:32:16, 32:32:16).

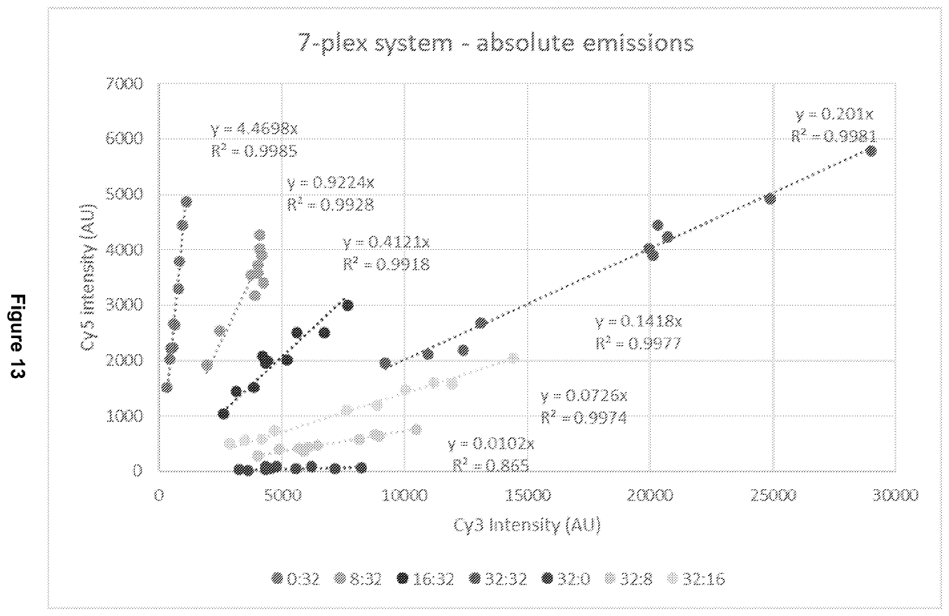

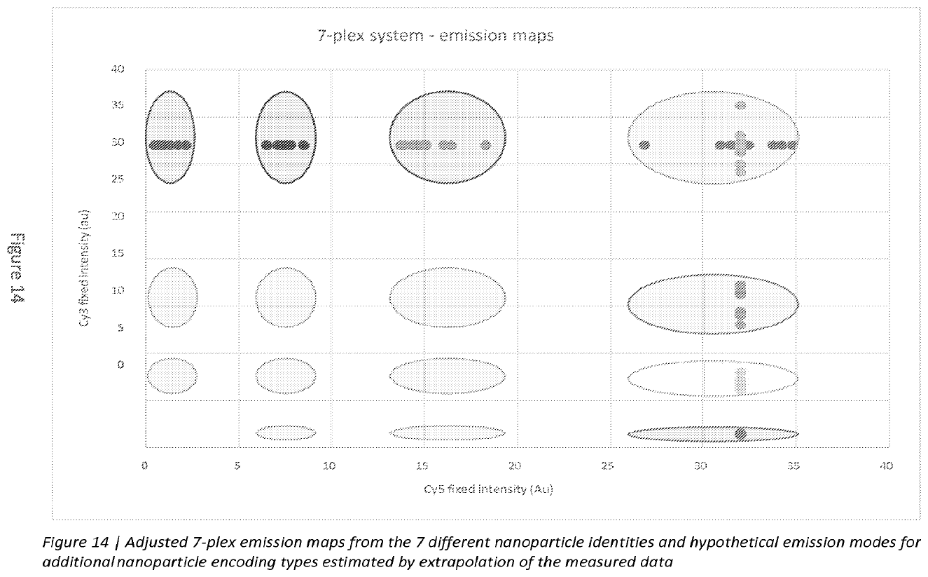

The absolute emissions of the 7-plex system can be seen in FIG. 13 and the corresponding emission map of the 7-plex system can be seen in FIG. 14.

Nanoparticle Coating

FIG. 3 shows a general schematic of coating the nanoparticles.

NP surface functionalization was performed by adding 100 μL of NPs (175 mM Si) to ethanol (248.5 μL), followed by H2O (226.5 μL). To this was added ammonium hydroxide solution (20 μL, 2.8%, 1:10 dilution in EtOH). Finally, 11-azidoundecyltriethoxysilane (10 μL, 1:4 dilution in THF) was added and the temperature raised to 37° C. during stirring. After 18 hours, the NPs were washed by centrifugation 3 times using THF. The N3-NPs were then redispersed in 100 μL THF and stored at 4° C. until further use.

Above functionalized N3-NPs (50 μL) were added to H₂O (112.6 μL) followed by DBCO modified oligo (4 μL, 100 μM, SEQ ID No:5). The temperature was raised to 37° C. After 1 hour, ADIBO-PEG4-acid (1.1 μL, 420 mM) was added. After 2 hours, the NPs were washed by centrifugation 3 times using EtOH. The NPs were then redispersed in 50 μL EtOH and stored at 4° C. until further use.

In another embodiment, above functionalized N3-NPs (50 μL) were added to H2O (112.6 μL) followed by DBCO modified oligo (4 μL, 100 μM, SEQ ID No:5) and ADIBO-PEG4-acid (0.3 μL, 115 mM). After 5 hours, the NPs were washed by centrifugation 3 times using EtOH. The NPs were then redispersed in 50 μL EtOH and stored at 4° C. until further use.

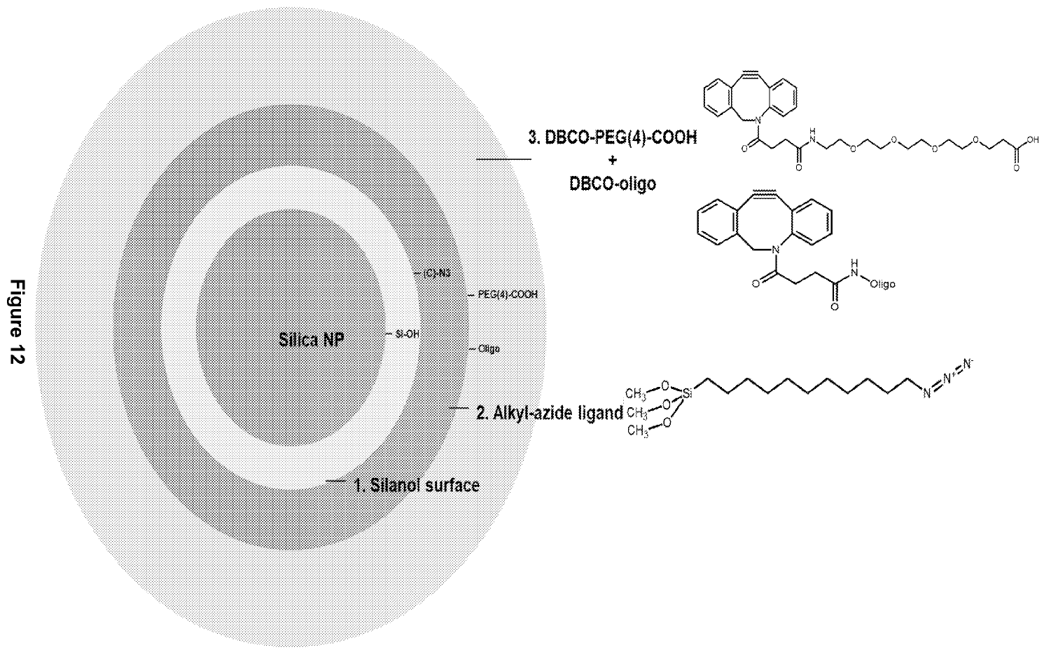

FIG. 12 is a schematic of the overall nanoparticle arrived at in this step.

In another embodiment, NP surface functionalization was performed by adding 100 μL of NPs (175 mM Si) to H2O (448 μL). To this was added ammonium hydroxide solution (2 μL, 28%) followed by mPEG5k-triethoxysilane (45 μL, 20 mM in H2O) and N3-PEG5k-triethoxysilane (5 ML, 10 mM in H2O). The temperature was raised to 75° C. during stirring or shaking. After 18 hours, the NPs were washed by centrifugation 3 times using H2O. The PEG-NPs were then redispersed in 100 μL H2O and stored at 4° C. until further use.

In another embodiment, NP surface functionalization was performed by adding 100 μL of NPs (175 mM Si) to H2O (448 ML). To this was added ammonium hydroxide solution (2 μL, 28%) followed by mPEG2k-triethoxysilane (45 ML, 20 mM in H2O) and N3-PEG5k-triethoxysilane (5 μL, 10 mM in H2O). The temperature was raised to 75° C. during stirring or shaking. After 18 hours, the NPs were washed by centrifugation 3 times using H2O. The PEG-NPs were then redispersed in 100 μL H2O and stored at 4° C. until further use.

Above functionalized PEG-NPs (30 ML) were added to H2O (5 ML) followed by DBCO modified oligo (5 μL, 100 μM, SEQ ID No:5). The temperature was raised to 37° C. After 18 hours, the NPs were washed by centrifugation 3 times using H2O. The NPs were then redispersed in 300 μL H2O and stored at 4° C. until further use.

What is particularly surprising is that when a PEG chain of 4000 Da or greater was used stability to the nanoparticle dispersion was provided, but the oligo was unable to bind to the target site. However, when using the shorted PEG4 chain, stability was still provided and the oligo was able to bind to the target site.

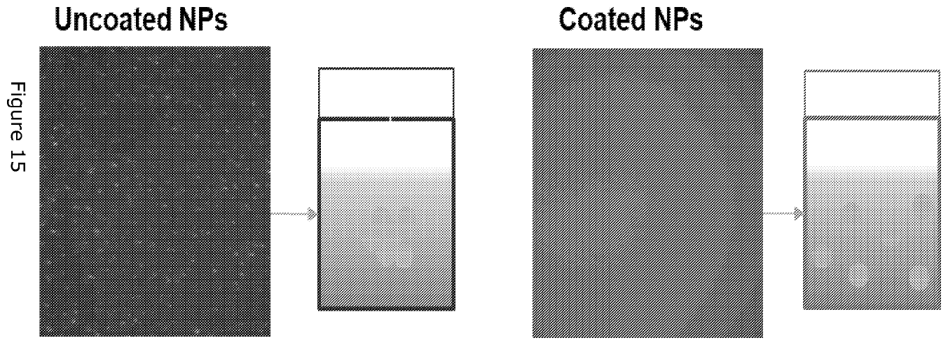

FIG. 15 shows how when nanoparticles have coatings that do not require the requisite stability they aggregate together and crash out of solution. Whereas when the coating is adequate they remain suspended and stable in the solution.

Nanoparticle to RCP Hybridization

RCP stock (30 μL) was diluted [1:5] in SSC 1× buffer (Invitrogen) by the addition of 120 μL SSC 1× buffer to a PCR tube (200 μL) containing the RCP solution. A circle was marked on Superfrost-plus slide (25×75×2 mm, VWR) using a diamond tip pen. Next, 4.5 μL of the diluted RCP solution was added as a drop on the marked circle, followed by drying in a 37° C. oven for 5 minutes. The marked circle with the dried RCPs was washed by pipetting 200 μL PBS-tween20 (0.01M, 0.05% tween20, Invitrogen), the washing step was repeated 2 times and the area surrounding the marked circle was cleaned using a microfiber tissue.

A hybridization chamber (Grace Bio-labs, Secure-seal hybridization chamber 8-9 mm Diameter×0.8 mm depth) was attached over the marked circle on the superfrost-plus slide. Next, the labelling solution was prepared. 130.6 μL H₂O was added to an Eppendorf tube (1.5 mL), 45 μL SSC buffer (4×, Invitrogen) followed by 10 seconds vortex of the solution at max setting. Next, 1.8 μL AF750-detection oligo (1 μM, SEQ ID No:11) was added followed by vortex for 10 seconds at max setting. The oligo functionalized nanoparticle stock prepared above was sonicated for 20 seconds. Oligo functionalized nanoparticle stock was added (2.5 μL) to the Eppendorf tube followed by 5 seconds vortex at max setting, 10 seconds sonication and 10 seconds vortex at max setting.

The prepared labelling solution was added to the hybridization chamber filling the chamber until full (45-50 μL). Adhesive plastic covers (3M VHB) were attached to the holes of the hybridization chamber. Next, the prepared slide was incubated for 1 hour in a oven at 37° C. After incubation, the plastic covers were removed using tweezers and the labelling solution was removed, emptying the hybridization chamber. The sample was then washed using the following procedure. 50 μL PBS-tween20 buffer was added to the hybridization chamber, the chamber was washed by removing and adding the liquid 10 times to the chamber using the pipette. The PBS-tween20 buffer was then discarded. The washing step was repeated 3×times.

Next, the hybridization chamber was detached form the superfrost-plus slide using tweezers. The slide was covered with a cardboard box and allowed to dry for 5 minutes in room temperature. Next, 7 μL slowfade (Gold antifade mountant, Invitrogen) was added to the marked circle and covered with coverglass (Menzel-Glaser 24×50 mm #1.5).

Image Acquisition

All fluorescent microscopy imaging was performed using a standard epifluorescent microscope (Zeiss Axio Imager.Z2) with an external LED light source (Lumencor SPECTRA X light engine). The microscope setup used a light engine with filter paddles (395/25, 438/29, 470/24, 555/28, 635/22, 730,50). Images were obtained with a sCMOS camera (2048×2048, 16 bit, ORCA-Flash4.0LT Plus, Hamamatsu) using objectives 20× (0.8 NA, air, 420650-9901) and 5× (0.16NA, air, 420630-9900). The setup used filter cubes for wavelength separation including quad band Chroma 89402 (DAPI, Cy3, Cy5) and quad band Chroma 89403 (Atto425, TexasRed, AlexaFluor750). All samples were mounted on an automatic multi-slide stage (PILine, M-686K011). The nanoparticles were imaged in the Cy3 and Cy5 channel using 100 ms exposure. The detection oligo was imaged in the AF750 channel using 200 ms exposure. The images were obtained using Z-stack with 5 μm height and 0.25 μm slice thickness resulting in 21 slices. All images were taken in ambient, dark microscopic room conditions.

Image Processing and Signal Decoding

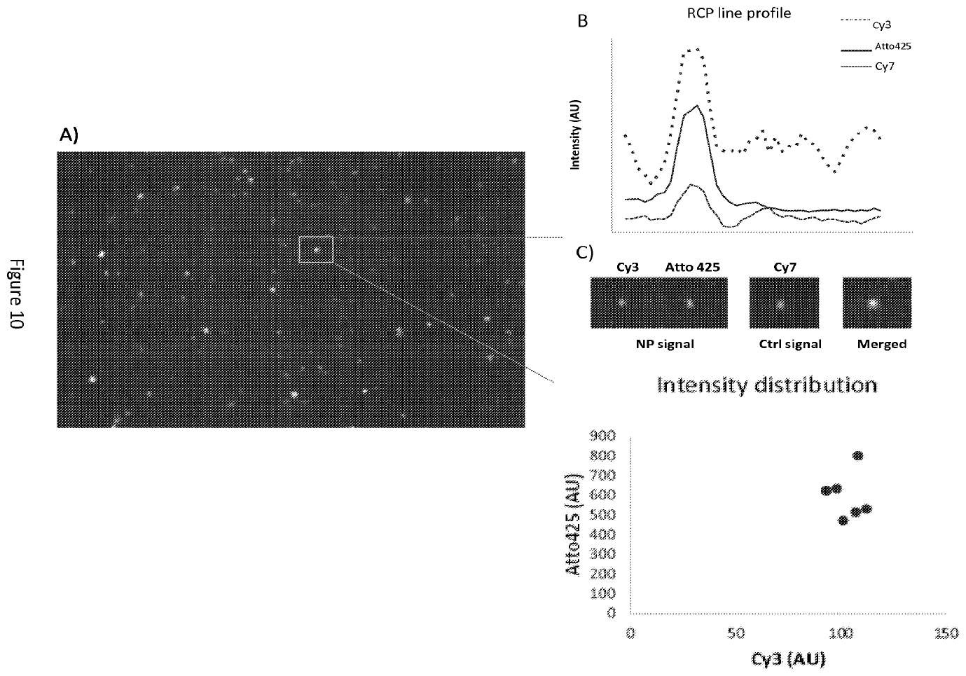

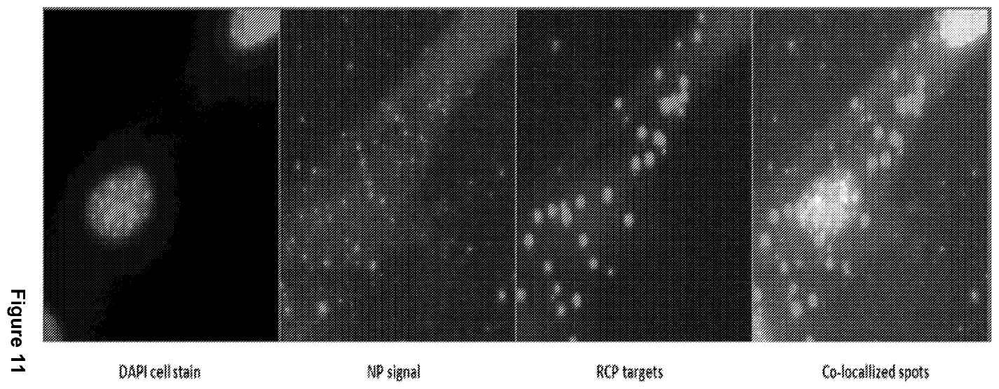

A typical image and signal decoding procedure is shown in FIG. 10. The images were analyzed using ZEN 3.2 (blue edition) software or ImageJ. In one embodiment, orthogonal projection was made using maximum projection method from the Z-stack images. In another embodiment, orthogonal projection was made using average projection method. In another embodiment, no orthogonal projection was made, instead the signal spot was segmented in 3-dimensions and the signal was analyzed in the 3-dimensional space.

FIG. 10(a) shows all channel combined orthogonal image. FIG. 10(b) shows the enlarged area which is corresponding to the white box shown in FIG. 10(a) and its individual channels (Cy3, Atto425 and Cy7 respectively). Cy3 and Atto425 fluorescence signal comes the nanoparticles, whereas Cy7 signal from the co-labelled detection oligos (DO). The colocalization between all these channels (NPs, DO) acts as a method to ensure NP binding to biomolecule and therefore can be used to filter out false positives. In this case false positives can be non-specifically bound particles or aggregates of particles.

In one embodiment, to decode the nanoparticle type identity, the fluorescence intensity/profile was measured in respective channel for a spatially resolved signal spot containing a cluster of nanoparticles bound to the biomolecule (RCP). The method of analyzing a single spot and optically decoding the nanoparticle type identity is performed by drawing a line profile over one spot shown magnified in FIG. 10(b). The emission wavelength (separated by the acquisition channels Cy3, Atto 425 and Cy7) as well as the intensity profiles from each channel is shown in FIG. 10(c) from such line profile. From these profiles, the maximum intensity values were measured for each channel and the corresponding background signal, which was averaged from the lower flat part of the intensity profile. In another embodiment, the mean background intensity (line profile) for each channel was averaged from the area where there were no signal spots found. The emission intensity was then measured by subtracting the background intensity from the maximum intensity. By analyzing the emission intensity for each wavelength, the identity of the nanoparticle type can be decoded. For the 7-plex system mentioned above incorporating Cy3 and Cy5 into the nanoparticles for the encoding, the emissions intensities from 10 spots of each nanoparticle type are plotted in FIG. 13 using the method described above.

In another embodiment, to decode the nanoparticle type identity, instead of drawing a line profile over one spot a circle was drawn around the spot. The max intensity was measured for each channel inside the circle and the background signal was subtracted by averaging the background signal with an equivalent spot placed on an area where there were no signal spots were found. In another embodiment, the background signal was averaged by using the min value inside the circle. In another embodiment, the background signal was averaged by drawing a second circle around the first circle, removing the pixel information from the first circle, and measuring the average intensity in the remaining pixel information from the larger circle.

Emission maps from the above mentioned 7-plex system can be plotted as shown in FIG. 14 if the nanoparticles contain a third emission color that can be used to reference the two other emission colors, or if the absolute emissions are calibrated prior to analysis. Furthermore, the emission map illustrates additional identities that can be used to generate 8 additional nanoparticle encodings that can be distinguished by extrapolating the performance of 7-plex system, leading to a total 15-plex system according to the formula 4²−1=15 (2 colors, 4 intensity levels, subtracting the dark mode).

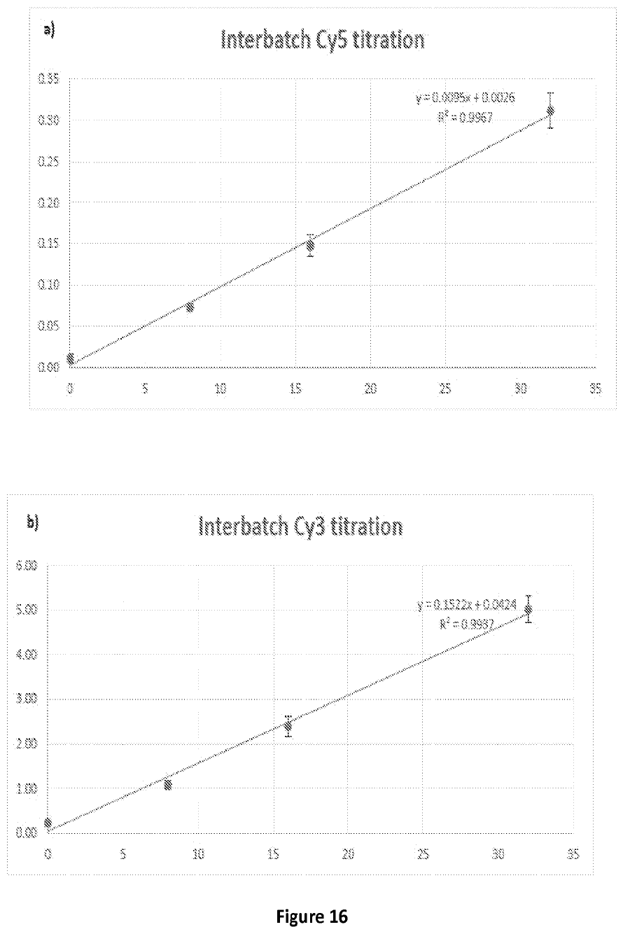

This is further supported by the titration plots shown in FIG. 16, where it is shown that the incorporation of each fluorophore can be controlled with high precision, meaning that any intensity level mapped out in FIG. 14 is achievable.

14-Plex Detection System with 7-Plex Nanoparticle Library Co-Labelled with 2-Plex Molecular Probes

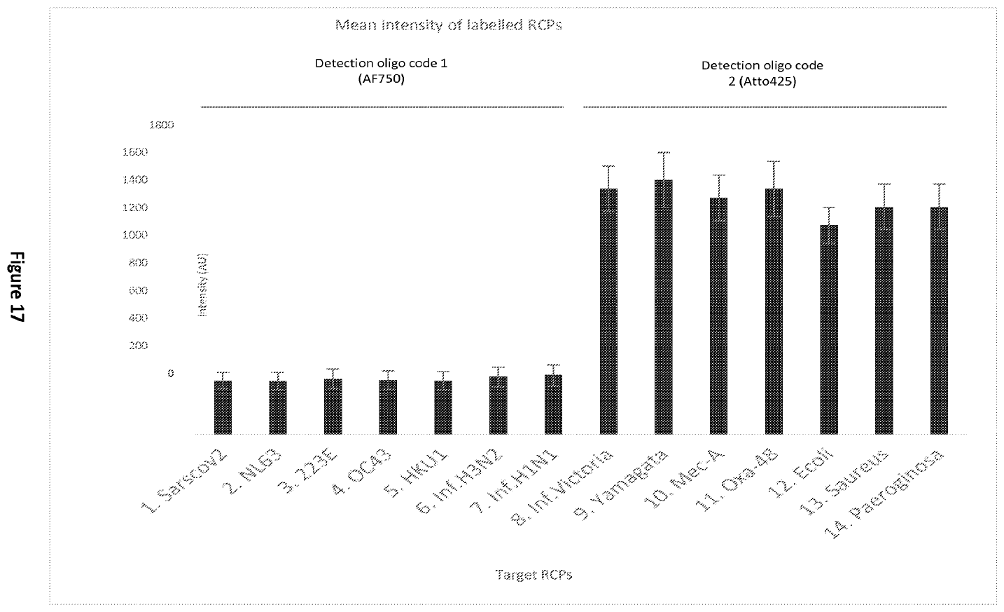

In another embodiment, a labelling solution was prepared with two detection oligos. 130.6 μL H2O was added to an Eppendorf tube (1.5 mL), 45 μL SSC buffer (4×, Invitrogen) followed by 10 seconds vortex of the solution at max setting. Next, 1.8 μL AF750-detection oligo (1 μM, SEQ ID No:11) and Atto425-detection oligo (1 μM, SEQ ID No: 12) was added followed by vortex for 10 seconds at max setting. The oligo functionalized nanoparticle stocks prepared above was sonicated for 20 seconds. Oligo functionalized nanoparticle stocks was added (7×2.5 μL) to the Eppendorf tube followed by 5 seconds vortex at max setting, 10 seconds sonication and 10 seconds vortex at max setting. The process of nanoparticle to RCP hybridization, image acquisition, processing and signal decoding was then applied according to above. The results are visualized in FIG. 13 and FIG. 14 for the nanoparticle decoding, and in FIG. 17 for the detection oligo decoding illustrating a 14-plex system established by combining a NP library of 7-plex co-labelled with detection oligo (molecular probe) library of 2-plex, using the same principles of signal decoding described above and shown in FIG. 10.

In another embodiment, a 30-plex system is achieved by combining a NP library of 15-plex as mentioned above co-labelled with detection oligo (molecular probe) library of 2-plex.

The described procedure for oligo functionalization and subsequent hybridization to RCPs used the following sequences listed in the table below.

| ID | | |
|---|---|---|
| No. | Nucleic acid type | |
| 5 | DBCO oligo (SARS-COV-2) | 5′-DBCO-TTTTTTTTTTTTCCTCAGTAATAGTGTCTTAC- |
| | | 3′ |
| 6 | DBCO oligo (HIV) | 5′-DBCO-TTTTTTTTTTTGCGTCTATTTAGTGGAGCC-3′ |
| 7 | Synthetic target HIV | CTCTCTCTCTCTATACTATATGTTTTAGTTTATATT |
| | | GTTTCTTTCCCCCTGGCCTTACCGAATTTT |
| | | TTCCCATTTATCTAATTCTCCCCCGCT |
| 8 | Synthetic target SARS-COV-2 | 5′-Biotin- |
| | | CTCTCTCTCTCTCTCTCTCTCTCTCTCTCTCGGT |
| | | GTGACAAGCTACAACACGTTGTATGTTTGCGAGCAA |
| 9 | Padlock Probe HIV | AAGGCCAGGGGGAAAG AGTAGCCGTGACTATCGACT |
| | | TGCGTCTATTTAGTGGAGCC |
| | | TTAAATGGGAAAAAATTCGGTT |
| 10 | Padlock probe SARS-COV-2 | TGTTGTAGCTTGTCACACCGGTGTATGCA |
| | | GCTCCTCAGTAATAGTGTCTTACGGCATCACTGGTTAC |
| | | GTCTGTTGCTCGCAAACATACAACG |
| 11 | AF750-Detection oligo (SARS- | 5′-TCCTCAGTAATAGTGTCTTACTTTT-AF750 |
| | COV-2) | |
| 12 | Atto425-Detection oligo | 5′-Atto425-CCTCAGTAATAGTGTCTTAC-3′ |

Nucleic acid sequences of DBCO oligos, synthetic targets and padlock probes used for procedures described in this document are listed in the table above.

1. A method for multiplexed detection of a plurality of target biomolecules using optical encoding, wherein each target biomolecule has at least one detection target, comprising the steps of:
a. providing a plurality of nanoparticle types comprising a plurality of nanoparticles, each nanoparticle having a coating that provides binding affinity of the nanoparticle to a type-specific detection target, and wherein each nanoparticle comprises a plurality of fluorophores that generates a signal which is unique for each nanoparticle type;
b. providing a sample comprising a plurality of target biomolecules;
c. contacting the sample with the plurality of nanoparticle types, thereby allowing the nanoparticles to bind with the detection targets of the target biomolecules; and
d. optically decoding the fluorophore signals emitted by the nanoparticle of the nanoparticle types bound to the detection target of the target biomolecules by measuring the wavelength and intensity of the emitted signals, thereby detecting the presence and identity of the target biomolecules.

2. The method according to claim 1, wherein each nanoparticle type is optically encoded by comprising controlled ratios of the plurality of fluorophores, thereby controlling the emission wavelength and intensity from the nanoparticle type, or (ii) altering the properties of the fluorophores affecting its emission intensity.

3. The method according to claim 1, wherein the binding affinity of the coating of the nanoparticle type is provided by a detection probe X attached via a linker to the nanoparticle, wherein the detection probe X is chosen from a nucleic acid molecule, an antigen or an antibody.

4. The method according to claim 1, wherein the coating of the nanoparticle furthermore comprises a repulsive component provided by a functional group Y attached via a linker to the nanoparticle, wherein the functional group Y is chosen from a charged group with a positive or negative charge, a zwitterionic group comprising both a positive or negative charge, or a sterically repulsive functional group.

5. The method according to claim 3, wherein the linker comprises at least one anchor group, which tethers the coating to the nanoparticle.

6. The method according to claim 3, wherein one or more linkers can provide one or more detection probes X and/or functional groups Y, or where multiple linkers can provide multiple detection probes X and/or functional groups Y via an interconnecting backbone.

7. The method according to claim 1, wherein the plurality of fluorophores are chosen from:
(i) organic fluorophores;
(ii) inorganic fluorophores; and/or
(iii) a combination of organic fluorophores and inorganic fluorophores as defined in (i) and (ii).

8. The method according to claim 1, wherein the plurality of nanoparticles are silica nanoparticles, semiconductor nanoparticles, organic nanoparticles, inorganic nanoparticles, metal nanoparticles or polymeric nanoparticles, or combinations thereof.

9. The method according to claim 1, wherein the plurality of nanoparticles have an average diameter of less than 300 nm.

10. The method according to claim 1, wherein the plurality of nanoparticles are porous nanoparticles.

11. The method according to claim 1, wherein prior to step c, the target biomolecules are prepared by binding them to at least one molecule comprising the detection target for subsequent amplification.

12. The method according to claim 1, wherein the detection target comprises a nucleic acid molecule, which is, or facilitates a molecule that is, amplified using RCA or multiple hybridization events.

13. The method according to claim 1, wherein the decoding is effected by optical decoding.

14. The method according to claim 1, further providing one or more molecular probes, wherein each molecular probe comprises a fluorophore that is bound to a nucleic acid molecule, an antigen or an antibody providing binding affinity of the molecular probe to the specific detection target.

15. A kit of parts, comprising, in separate containers,
(i) a plurality of nanoparticle(s) types comprising a plurality of nanoparticles, wherein each nanoparticle comprises a plurality of fluorophores that generates a signal which is unique for each nanoparticle type,
(ii) a probing buffer, comprising a solution with controlled pH, salt concentration and additives facilitating specific detection target binding of the nanoparticle(s); and
(iii) instructions for use of the kit in the method according to claim 1.

16. The method according to claim 5, wherein the linker further comprises a spacer group.

17. The method according to claim 7, wherein the organic fluorophores are selected from the list consisting of Atto 425, Alex fluor 405, Alexa Fluor 488, fluorescin, DiO, Atto 488, Cy3, DiI, Alexa fluor 546, Atto 550, Cy5, Alexa fluor 647, Texas red, DiD, Atto647(N), Atto 655, Cy7, Alexa fluor 680, Alexa fluor 750, Atto 680, and Atto 700, and combinations thereof.

18. The method according to claim 7, wherein the inorganic fluorophores are selected from the list consisting of quantum dots, rods, perovskite quantum dots, and metal-ligand complexes, and combinations thereof.

19. The method according to claim 1, wherein the plurality of nanoparticles have an average diameter of 3 to 100 nm.

20. The method according to claim 1, wherein the optical decoding is optical imaging.


SEQUENCE LISTING (12 sequences)

SEQ ID NO: 1 - DNA, 30 bp, Artificial Sequence
  1
ttttttttttcctcagtaatagtgtcttac

SEQ ID NO: 2 - DNA, 96 bp, Artificial Sequence
  2
ttacaccctttctttgacaagtgtatgcagctcctcagtaatagtgtctttgtgcgtctatttagtggagcccatggactgttactgagctgcgtt

SEQ ID NO: 3 - DNA, 40 bp, Artificial Sequence
  3
ttgtcaaagaaagggtgtaaaacgcagctcagtaacagtc

SEQ ID NO: 4 - DNA, 20 bp, Artificial Sequence
  4
tgcgtctatttagtggagcc

SEQ ID NO: 5 - DNA, 32 bp, Artificial Sequence
  Comprises 5′-Dibenzocyclooctyne (DBCO); DBCO oligo (SARS-COV-2)
  misc_feature 1
ttttttttttttcctcagtaatagtgtcttac

SEQ ID NO: 6 - DNA, 30 bp, Artificial Sequence
  Comprises 5′-Dibenzocyclooctyne (DBCO); DBCO oligo (HIV)
  misc_feature 1
tttttttttttgcgtctatttagtggagcc

SEQ ID NO: 7 - DNA, 94 bp, Artificial Sequence
  Synthetic target HIV
ctctctctctctatactatatgttttagtttatattgtttctttccccctggccttaaccgaattttttcccatttatctaattctcccccgct

SEQ ID NO: 8 - DNA, 70 bp, Artificial Sequence
  Comprises 5′-Biotin; Synthetic target SARS-COV-2
  misc_feature 1
ctctctctctctctctctctctctctctctcggtgtgacaagctacaacacgttgtatgtttgcgagcaa

SEQ ID NO: 9 - DNA, 78 bp, Artificial Sequence
  Comprises 5′-phosphorylation (PO4); Padlock Probe HIV
  misc_feature 1
aaggccagggggaaagagtagccgtgactatcgacttgcgtctatttagtggagccttaaatgggaaaaaattcggtt

SEQ ID NO: 10 - DNA, 92 bp, Artificial Sequence
  Comprises 5′-phosphorylation (PO4); Padlock probe SARS-COV-2
  misc_feature 1
tgttgtagcttgtcacaccggtgtatgcagctcctcagtaatagtgtcttacggcatcactggttacgtctgttgctcgcaaacatacaacg

SEQ ID NO: 11 - DNA, 25 bp, Artificial Sequence
  Comprises 3′-AF750; AF750-Detection oligo (SARS-COV-2)
  misc_feature 25
tcctcagtaatagtgtcttactttt

SEQ ID NO: 12 - DNA, 20 bp, Artificial Sequence
  Comprises 5′-Atto425; Atto425-Detection oligo
  misc_feature 1
  misc_feature 1
cctcagtaatagtgtcttac